(19) United States
(12) Patent Application Publication
(10) Pub. No.: US 2026/0157415 A1
(43) Pub. Date: Jun. 11, 2026

(54) ORAL POUCH PRODUCT

(71) Applicant: Altria Client Services LLC, Richmond, VA (US)

(72) Inventors: Shaoyong YU, Glen Allen, VA (US); Shuzhong ZHUANG, Glen Allen, VA (US); Baiwei CHEN, Glen Allen, VA (US); Shannon BLACK, Richmond, VA (US); Feng GAO, Midlothian, VA (US); Benjamin L. RAGLAND, Henrico, VA (US); Rebecca GRAY, Richmond, VA (US); Fadi ALDEEK, Glen Allen, VA (US)

(73) Assignee: Altria Client Services LLC, Richmond, VA (US)

(21) Appl. No.: 19/406,410

(22) Filed: Dec. 2, 2025

Related U.S. Application Data
(60) Provisional application No. 63/728,996, filed on Dec. 6, 2024.

(51) Int. Cl. A24B 15/16; A24B 13/00; A24B 15/32; A24B 15/40; A61K 9/00; A61K 31/465

(52) U.S. Cl. CPC A24B 15/16; A24B 13/00; A24B 15/32; A24B 15/403; A61K 9/009; A61K 31/465

(57) ABSTRACT

An oral pouch product includes a wrapper and a filler. The wrapper defines a cavity. The filler is in the cavity. The filler includes a dry mixture and a liquid mixture. The dry mixture includes a cellulosic material and a water-soluble filler. The liquid mixture includes oil, water, and liquid nicotine. The oil includes a triglyceride, a diglyceride, a monoglyceride, or any combination thereof. The liquid nicotine is dissolved in the oil and the water.

### CROSS-REFERENCE TO RELATED APPLICATIONS

This application claims the benefit of U.S. Provisional Application No. 63/728,996 filed on Dec. 6, 2024, the entire disclosure of which is incorporated herein by reference in its entirety.

### BACKGROUND

The present disclosure relates to oral pouch products including nicotine.

Oral nicotine products are available in a variety of formats, such as gums, sprays, lozenges, dissolvable tables, non-dissolvable chews, films, gels, capsules, sticks (e.g., coated wooden dowels or singular dissolvable sticks), and pouches (e.g., containing fibers or granules). Oral pouch products may have nicotine levels that create a familiar experience for adult tobacco consumers.

### SUMMARY

At least one example embodiment relates to an oral pouch product.

In at least one example embodiment, the oral pouch product includes a wrapper defining a cavity and a filler in the cavity. The filler includes a dry mixture and a liquid mixture. The liquid mixture includes oil. The liquid mixture includes water in an amount of 10 weight percent or more based on a weight of the filler.

In at least one example embodiment, the water content is between 10 and 30 weight percent based on the weight of the filler.

In at least one example embodiment, the water content is between 20 and 30 weight percent based on the weight of the filler.

In at least one example embodiment, the oral pouch product further includes an additive.

In at least one example embodiment, the additive includes liquid nicotine.

In at least one example embodiment, the oral pouch product is configured to release at least 30 weight percent of the liquid nicotine based on a total weight of nicotine in the oral pouch product within 10 minutes upon placement in a mouth.

In at least one example embodiment, the additive includes a sweetener, a pH adjuster, a flavorant, a humectant, or any combination thereof.

In at least one example embodiment, a density of the filler is about 0.3 g/cm³.

In at least one example embodiment, the oil includes a medium chain triglyceride.

In at least one example embodiment, the dry mixture includes a cellulosic material.

In at least one example embodiment, the dry mixture includes microcrystalline cellulose.

In at least one example embodiment, the filler includes between 30 weight percent to 60 weight percent dry mixture.

In at least one example embodiment, the filler includes more than 20 weight percent oil.

In at least one example embodiment, the filler includes more than 20 weight percent oil and between 10 weight percent and 30 weight percent water based on the weight of the filler.

In at least one example embodiment, the filler includes a medium chain triglyceride, water, microcrystalline cellulose, and liquid nicotine.

In at least one example embodiment, the filler includes more than 20 weight percent oil, between 10 weight percent and 30 weight percent water; and between 30 weight percent and 60 weight percent dry mixture.

In at least one example embodiment, the dry mixture is insoluble in the liquid mixture.

In at least one example embodiment, the oral pouch product includes a wrapper defining a cavity and a filler in the cavity. The filing material includes a liquid mixture and a dry mixture. The dry mixture includes between 30 weight percent and 60 weight percent microcrystalline cellulose based on a weight on the pouch product. The liquid mixture includes more than 20 weight percent medium chain triglyceride based on the weight of the filler; water in an amount between 10 weight percent and 30 weight percent based on the weight of the filler; and liquid nicotine.

In at least one example embodiment, the oral pouch product is configured to release at least 30 weight percent of the liquid nicotine based on a total weight of nicotine in the oral pouch product within 10 minutes upon placement in a mouth.

In at least one example embodiment the oral pouch product includes a sweetener, a pH adjuster, a flavorant, a humectant, or any combination thereof.

In at least one example embodiment, a method for preparing a filler for an oral pouch product includes mixing an active ingredient solution with oil to form an oil pre-blend; preparing a dry pre-blend including an insoluble material and a dry flavorant; combining the oil pre-blend with the dry pre-blend to form a first mixture; and adding a water solution including a water-based flavorant into the first mixture to form a filler, the water in the water solution added in an amount of 10 weight percent or more based on a weight of the filler.

In at least one example embodiment, the oil pre-blend additionally includes an antioxidant.

In at least one example embodiment, the dry pre-blend additionally includes a water-soluble filler.

In at least one example embodiment, the dry pre-blend includes at least one of a pH adjuster, a dry antioxidant, plant material, an emulsifier, a surfactant, or any combination thereof.

In at least one example embodiment, the oil pre-blend is combined with the dry pre-blend in the form of an atomized spray.

In at least one example embodiment, the method further includes filling a pouch with the filler; and sealing the pouch to contain the filler and form the oral pouch product.

In at least one example embodiment, the method further includes forming the active ingredient solution by dissolving an active ingredient in oil in an amount greater than or equal to a weight ratio of 20:80 active ingredient to oil and less than our equal to a weight ratio of 80:20 active ingredient to oil.

In at least one example embodiment, the oil is a medium chain triglyceride.

In at least one example embodiment, the oil is included in an amount greater than or equal to 20 weight percent of the filler.

In at least one example embodiment, the dry mixture is included in an amount greater than or equal to 30 weight percent of the filler and less than or equal to 60 weight percent of the filler.

### BRIEF DESCRIPTION OF THE DRAWINGS

The various features and advantages of the non-limiting embodiments herein may become more apparent upon review of the detailed description in conjunction with the accompanying drawings. The accompanying drawings are merely provided for illustrative purposes and should not be interpreted to limit the scope of the claims. The accompanying drawings are not to be considered as drawn to scale unless explicitly noted. For purposes of clarity, various dimensions of the drawings may have been exaggerated.

FIG. 1A is a perspective view of an oral pouch product according to at least one example embodiment.

FIG. 1B is a cross-sectional view of the oral pouch product taken along line I-I of FIG. 1A according to at least one example embodiment.

FIG. 2A is a perspective view of an oral pouch product according to at least one example embodiment.

FIG. 2B is a cross-sectional view of the oral pouch product along line II-II of FIG. 2A according to at least one example embodiment.

FIG. 2C is a cross-sectional view of the oral pouch product along line III-III of FIG. 2A according to at least one example embodiment.

FIG. 3A is a side view of an oral pouch product according to at least one example embodiment.

FIG. 3B is a cross-sectional view along line VII-VII of the oral pouch product of FIG. 3A according at least one example embodiment.

FIG. 4A is a chemical structure of free-base nicotine.

FIG. 4B is a chemical structure of mono-protonated nicotine.

FIG. 4C is a chemical structure of di-protonated nicotine.

FIGS. 5A-5B are a graphs depicting buccal nicotine disposition for different nicotine solutions.

FIG. 6 is a graph depicting nicotine release profiles of different oil, water, and nicotine solutions.

FIG. 7A is a table depicting partition coefficient data for nicotine in different oil and aqueous phases.

FIG. 7B depicts a chemical structure of triacetin (C2).

FIG. 7C depicts a chemical structure of MCT (C8-C10).

FIG. 7D depicts a chemical structure of triolein (C18).

FIG. 8 is a flowchart depicting a method of preparing an oral pouch product according to at least one example embodiment.

### DETAILED DESCRIPTION

Some detailed example embodiments are disclosed herein. However, specific structural and functional details disclosed herein are merely representative for purposes of describing example embodiments. Example embodiments may, however, be embodied in many alternate forms and should not be construed as limited to only the example embodiments set forth herein.

Accordingly, while example embodiments are capable of various modifications and alternative forms, example embodiments thereof are shown by way of example in the drawings and will herein be described in detail. It should be understood, however, that there is no intent to limit example embodiments to the particular forms disclosed, but to the contrary, example embodiments are to cover all modifications, equivalents, and alternatives falling within the scope of example embodiments. Like numbers refer to like elements throughout the description of the figures.

It should be understood that when an element or layer is referred to as being “on,” “connected to,” “coupled to,” or “covering” another element or layer, it may be directly on, connected to, coupled to, or covering the other element or layer or intervening elements or layers may be present. In contrast, when an element is referred to as being “directly on,” “directly connected to,” or “directly coupled to” another element or layer, there are no intervening elements or layers present. Like numbers refer to like elements throughout the specification. As used herein, the term “and/or” includes any and all combinations of one or more of the associated listed items.

It should be understood that, although the terms first, second, third, etc., may be used herein to describe various elements, regions, layers and/or sections, these elements, regions, layers, and/or sections should not be limited by these terms. These terms are only used to distinguish one element, region, layer, or section from another region, layer, or section. Thus, a first element, component, region, layer, or section discussed below could be termed a second element, region, layer, or section without departing from the teachings of example embodiments.

Spatially relative terms (e.g., “beneath,” “below,” “lower,” “above,” “upper,” “inside,” “outside,” and the like) may be used herein for ease of description to describe one element or feature's relationship to another element(s) or feature(s) as illustrated in the figures. It should be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the term “below” may encompass both an orientation of above and below. The device may be otherwise oriented (rotated 90 degrees or at other orientations) and the spatially relative descriptors used herein interpreted accordingly.

The terminology used herein is for the purpose of describing various example embodiments only and is not intended to be limiting of example embodiments. As used herein, the singular forms “a,” “an,” and “the” are intended to include the plural forms as well, unless the context clearly indicates otherwise. It will be further understood that the terms “includes,” “including,” “comprises,” and/or “comprising,” specify the presence of stated features, integers, steps, operations, and/or elements, but do not preclude the presence or addition of one or more other features, integers, steps, operations, elements, and/or groups thereof.

Example embodiments are described herein with reference to cross-sectional illustrations that are schematic illustrations of example embodiments. As such, variations from the shapes of the illustrations are to be expected. Thus, example embodiments should not be construed as limited to the shapes of regions illustrated herein but are to include deviations and variations in shapes. When the terms “about” or “substantially” are used in connection with a numerical value, it is intended that the associated numerical value include a tolerance of +10% around the stated numerical value unless the context indicates otherwise.

Unless otherwise defined, all terms (including technical and scientific terms) used herein have the same meaning as commonly understood by one of ordinary skill in the art to which example embodiments belong. It will be further understood that terms, including those defined in commonly used dictionaries, should be interpreted as having a meaning that is consistent with their meaning in the context of the relevant art and will not be interpreted in an idealized or overly formal sense unless expressly so defined herein.

In at least one example embodiment, methods of enhancing flavor and/or sensory effects of nicotine in oral pouch products, such as oral pouch products including nicotine and cellulose in a pouch wrapper are provided.

In at least one example embodiment, the oral pouch product is an oral tobacco product, an oral non-tobacco product, an oral cannabis product, or any combination thereof. The oral pouch product may be in a form of loose material (e.g., loose cellulosic material), shaped material (e.g., plugs or twists), pouched material, tablets, lozenges, chews, gums, films, any other oral pouch product, or any combination thereof.

The oral pouch product may include chewing tobacco, snus, moist snuff tobacco, dry snuff tobacco, other smokeless tobacco and non-tobacco products for oral consumption, or any combination thereof.

Where the oral pouch product is an oral tobacco product including smokeless tobacco product, the smokeless tobacco product may include tobacco that is whole, shredded, cut, granulated, reconstituted, cured, aged, fermented, pasteurized, or otherwise processed. Tobacco may be present as whole or portions of leaves, flowers, roots, stems, extracts (e.g., nicotine), or any combination thereof.

In at least one example embodiment, the oral pouch product includes a tobacco extract, such as a tobacco-derived nicotine extract, and/or synthetic nicotine. The oral pouch product may include nicotine alone or in combination with a carrier (e.g., white snus), such as a cellulosic material. The carrier may be a non-tobacco material (e.g., microcrystalline cellulose) or a tobacco material (e.g., tobacco fibers having reduced or eliminated nicotine content, which may be referred to as “exhausted tobacco plant tissue or fibers”). In some example embodiments, the exhausted tobacco plant tissue or fibers can be treated to remove at least 25%, 40%, 50%, 60%, 70%, 75%, 80%, 85%, 90%, or 95% of the nicotine. For example, the tobacco plant tissue can be washed with water or another solvent to remove the nicotine.

In other example embodiments, the oral pouch product may include cannabis, such as cannabis plant tissue and/or cannabis extracts. In at least one example embodiment, the cannabis material includes leaf and/or flower material from one or more species of cannabis plants and/or extracts from the one or more species of cannabis plants. The one or more species of cannabis plants may include Cannabis sativa, Cannabis indica, and/or Cannabis ruderalis. In at least one example embodiment, the cannabis may be in the form of fibers. In at least one example embodiment, the cannabis may include a cannabinoid, a terpene, and/or a flavonoid. In at least one example embodiment, the cannabis material may be a cannabis-derived cannabis material, such as a cannabis-derived cannabinoid, a cannabis-derived terpene, and/or a cannabis-derived flavonoid.

The oral pouch product (e.g., the oral tobacco product, the oral non-tobacco product, or the oral cannabis product) may have various ranges of moisture. In at least one example embodiment, the oral pouch product is a dry oral pouch product having a moisture content ranging from 5% by weight to 10% by weight. In at least one example embodiment, the oral pouch product has a medium moisture content, such as a moisture content ranging from 20% by weight to 35% by weight. In at least one example embodiment, the oral pouch product is a wet oral pouch product having a moisture content ranging from 40% by weight to 55% by weight.

The oral pouch product may be sized and/or shaped and/or configured to be wholly received in an oral cavity of an adult tobacco consumer. The oral pouch product may have an oval shape, a rounded shield shape, a flat shield shape an elliptical shape, an elongated elliptical shape, a semi-circular shape, a square shape, a rounded-edge square shape, a rectangular shape, an elongated rectangular shape, a rounded-edge rectangular shape, a football shape, a boomerang shape, a teardrop shape, a comma shape, a bowtie shape, a peanut shape, or any combination thereof. The oral pouch product may have an oral-shaped cross section, a rectangular cross section, an elongated rectangular cross section, a lens or football shaped cross section, a boomerang-shaped cross section, a shield-shaped cross section, or any combination thereof. In at least one example embodiment, the size and/or shape and/or configuration of the oral pouch product may be selected to promote desired positioning of the oral pouch product within the oral cavity and/or packaging.

In at least one example embodiment, the oral pouch product may have dimensions ranging from about 1 millimeter to about 25 millimeters (e.g., about 1 millimeter to about 10 millimeters, about 1 millimeter to about 5 millimeters, about 5 millimeters to about 25 millimeters, about 5 millimeters to about 10 millimeters, about 10 millimeters to about 15 millimeters, about 15 millimeters to about 20 millimeters, or about 20 millimeters to about 25 millimeters). In at least one example embodiment, the oral pouch product has a first dimension (e.g., smallest dimension or thickness) ranging from about 1 millimeter to about 10 millimeters (e.g., about 2.5 millimeters). In at least one example embodiment, the oral pouch product has a largest dimension (e.g., diameter, height, or width) ranging from about 5 millimeters to about 25 millimeters (e.g., about 12 millimeters).

In at least one example embodiment, the oral pouch product may have a weight ranging from about 1 gram to about 10 grams (e.g., about 1 gram to about 9 grams, about 1 gram to about 8 grams, about 1 gram to about 7 grams, about 1 gram to about 6 grams, about 1 gram to about 5 grams, about 1 gram to about 4 grams, about 1 gram to about 3 grams, about 1 gram to about 2 grams, about 2 grams to about 9 grams, about 2 grams to about 8 grams, about 2 grams to about 7 grams, about 2 grams to about 6 grams, about 2 grams to about 5 grams, about 2 grams to about 4 grams, about 2 grams to about 3 grams, about 3 grams to about 9 grams, about 3 grams to about 8 grams, about 3 grams to about 7 grams, about 3 grams to about 6 grams, about 3 grams to about 5 grams, about 3 grams to about 4 grams, about 4 grams to about 9 grams, about 4 grams to about 8 grams, about 4 grams to about 7 grams, about 4 grams to about 6 grams, about 4 grams to about 5 grams, about 5 grams to about 9 grams, about 5 grams to about 8 grams, about 5 grams to about 7 grams, about 5 grams to about 6 grams, about 6 grams to about 9 grams, about 6 grams to about 8 grams, about 6 grams to about 7 grams, about 7 grams to about 9 grams, about 7 grams to about 8 grams, about 8 grams to about 9 grams).

### Additives

In at least one example embodiment, oral pouch product may include one or more elements such as a mouth-stable polymer, a mouth-soluble polymer, a sweetener (e.g., a synthetic sweetener and/or a natural sweetener), an energizing agent, a soothing agent (e.g., theanine and/or melatonin), a focusing agent (e.g., gingko biloba), a plasticizer, mouth-soluble or partially-soluble fibers (e.g., sugar beet fibers), an alkaloid, a mineral, a vitamin, a dietary supplement, a nutraceutical, a coloring agent, an amino acid, a chemesthetic agent, an antioxidant, a food-grade emulsifier, a pH modifier, a botanical (e.g., green tea), a tooth-whitening agent (e.g., sodium hexametaphosphate (SHMP)), a therapeutic agent, a processing aid, a stearate (e.g., magnesium and/or potassium), a wax (e.g., glycerol monostearate, propylene glycol monostearate, and/or an acetylated monoglyceride), a stabilizer (e.g., ascorbic acid and monosterol citrate, butylated hydroxytoluene (BHT), or butylated hydroxyanisole (BHA)), a lubricant (e.g., sodium lauryl sulfate (SLS)), a disintegrating agent, a lubricant, a preservative (e.g., sodium benzoate), a filler, a flavorant, an effervescent (e.g., carbon dioxide embedded in a flavorant or a filler), flavor masking agents, a bitterness receptor site blocker, a receptor site enhancers, other additives, or any combination thereof. The oral pouch product may include multiple additional elements. Additionally, a single element may belong to more than one of the categories above.

As used herein, “mouth-soluble” means that the polymer experiences significant degradation when exposed to saliva within an oral cavity of an adult consumer over a period of about four hours. In at least one example embodiment, the mouth-soluble polymer disintegrates when exposed to saliva having a normal human body temperature (i.e., 98.6° F.) for a period of less than or equal to about an hour (e.g., less than or equal to about 30 minutes, less than or equal to about 15 minutes, less than or equal to about 10 minutes, or less than or equal to about 5 minutes).

In at least one example embodiment, the oral pouch product includes a mouth-soluble polymer. The mouth-soluble polymer may include, for example, a cellulosic polymer, a natural polymer, a seaweed-derived polymer, a microbial-derived polymer, an extract, an exudate, a synthetic polymer, or any combination thereof. Other useful mouth-soluble polymers are known in the art, for example, see Krochta et al., Food Technology (1997) at 51:61-74; Glicksman Food Hydrocolloids CRC 1982; Krochta Edible Coatings and Films to Improve Food Quality Technomic 1994; Industrial Gums Academic 1993; and/or Nussinovitch Water-Soluble Polymer Applications in Foods Blackwell Science 2003, the entire contents of which are hereby incorporated by reference.

In at least one example embodiment, the cellulosic polymer may include, for example, carboxymethyl cellulose (CMC), hydroxypropyl (HPC), hydroxyethyl cellulose (HEC), hydroxypropyl methylcellulose (HPMC), methyl cellulose (MC), or any combination thereof. In at least one example embodiment, the natural polymer may include, for example, a starch, a modified starch, konjac, collagen, inulin, soy protein, whey protein, casein, wheat gluten, or any combination thereof. In at least one example embodiment, the seaweed-derived polymer may include, for example, a carrageenan, an alginate, or a combination of a carrageenan and an alginate. In at least one example embodiment, the carrageenan may include, for example, kappa carrageenan, iota carrageenan, lambda carrageenan, or any combination thereof. In at least one example embodiment, the alginate may include, for example, propylene glycol alginate. In at least one example embodiment, the microbial-derived polymer may include, for example, xanthan, dextran, pullulan, curdlan, gellan, or any combination thereof. In at least one example embodiment, the extract may include, for example, locust bean gum, guar gum, tara gum, gum tragacanth, pectin (e.g., low methoxy and amidated), agar, zein, karaya, gelatin, psyllium seed, chitin, chitosan, or any combination thereof. In at least one example embodiment, the exudate may include, for example, gum acacia (arabic), shellac, or any combination thereof. In at least one example embodiment, the synthetic polymer may include, for example, polyvinyl pyrrolidone, polyethylene oxide, polyvinyl alcohol, or any combination thereof.

As used herein, “mouth-stable” means that the polymer does not appreciably dissolve or disintegrate when exposed to saliva at the normal human body temperature (i.e., 98.6° F.) over a period of about one hour. In at least one example embodiment, the mouth-stable polymer is a biodegradable polymer that is configured to break down over a period of days, weeks, months, or years but does not appreciably break down when held in an oral cavity and exposed to saliva for a period of about one hour. In at least one example embodiment, the mouth-stable polymer is stable within an oral cavity and exposed to saliva at the normal human body temperature for a period of greater than or equal to about 2 hours (e.g., greater than or equal to about 6 hours, greater than or equal to about 12 hours, greater than or equal to about 1 day, or greater than or equal to about 2 days). Accordingly, an oral pouch product including a mouth-stable polymer according to at least one example embodiment is configured to remain intact when placed in an adult consumer's mouth. After a period of time, the mouth-stable polymer and any other mouth-stable elements may be removed from the adult consumer's mouth and discarded.

The mouth-stable polymer may be biocompatible and biostable. The mouth-stable polymer may generally be recognized as safe and in compliance with applicable food-contact regulations by an appropriate regulatory agency (e.g., the U.S. Food and Drug Administration (FDA)). In at least one example embodiment, the mouth-stable polymer has a flexural modulus of greater than or equal to about 5 MPa (e.g., greater than or equal to about 10 MPa) when tested according to ASTM Testing Method D790 or ISO 178 at 23° C.

In at least one example embodiment, the mouth-stable polymer may include, for example, a polyurethane, a silicone, a polyester, a polyacrylate, a polyethylene, a polypropylene, a polyetheramide, a polystyrene, a polyvinyl alcohol, a polyvinyl acetate, a polyvinyl chloride, a polybutyl acetate, a butyl rubber, poly(styrene-ethylene-butylene-styrene) (SEBS), poly(styrene-butadiene-styrene) (SBS), poly(styrene-isoprene-styrene) (SIS), any copolymer thereof, or any combination thereof. In at least one example embodiment, the mouth-stable polymer includes a food-grade or medical-grade polymer, such as medical-grade polyurethane.

In at least one example embodiment, the mouth-stable polymer includes, for example, a thermoplastic polymer. The thermoplastic polymer may include a thermoplastic elastomer. In at least one example embodiment, the mouth-stable polymer includes a thermoplastic elastomer meeting the requirements of the FDA-modified ISO 10993, Part 1 “Biological Evaluation of Medical Devices” tests with human tissue contact time of 30 days or less. In at least one example embodiment, the mouth-stable polymer has a shore Hardness of 50D or softer, a melt flow index of about 3 g/10 min at 200° C./10 kg, a tensile strength of greater than or equal to about 10 MPa (using ISO 37), and/or an ultimate elongation of less than about 100% (using ISO 37).

In at least one example embodiment, the oral pouch product is free of a sweetener. In at least one example embodiment, the oral pouch product includes a sweetener. In at least one example embodiment, the oral pouch product includes an encapsulated sweetener. The sweetener may include a synthetic sweetener, a natural sweetener, or a combination of a synthetic sweetener and a natural sweetener.

The natural sweetener may include, for example, a sugar such as monosaccharide, a disaccharide, a polysaccharide, or any combination thereof. The natural sweetener may include, for example, sucrose, honey, a mixture of low-molecular-weight sugars excluding sucrose, glucose (i.e., grape sugar, corn sugar, dextrose), molasses, corn sweetener, glucose syrup (i.e., corn syrup), fructose (i.e., fruit sugar), lactose (i.e., milk sugar), maltose (i.e., malt sugar, maltobiose), sorghum syrup, fruit juice concentrate, or any combination thereof.

In at least one example embodiment, the sweetener includes a sugar alcohol. The sugar alcohol may include, for example, ethylene glycol, glycerol, erythritol, threitol, arabitol, xylitol, ribitol, mannitol, sorbitol, galactitol, fucitol, iditol, inositol, volemitol, isomalt, maltitol, lactitol, maltotriitol, maltotetraitol, polyglycitol, or any combination thereof. In at least one example embodiment, the sweetener includes a non-nutritive sweetener. The non-nutritive sweetener may include, for example, stevia, saccharin, aspartame, sucralose, acesulfame potassium, or any combination thereof.

As used herein, the term “nutraceuticals” refers to any ingredient in foods that has a beneficial effect on human health. Nutraceuticals include particular compounds and/or compositions isolated from natural food sources and genetically modified food sources. Suitable nutraceuticals include, without limitation, various phytonutrients derived from natural plants and genetically engineered plants. The nutraceuticals can be included in an amount of about 0.1% to about 5% by weight based on the weight of the composition.

In at least one example embodiment, the oral pouch product may include the energizing agent. In at least one example embodiment, the energizing agent includes caffeine, taurine, glucaronalactone, guarana, vitamin B6, vitamin B12, or any combination thereof.

Caffeine, also known as 1,3,7-trimethylxanthine, is a white, odorless, bitter tasting substance. Caffeine occurs naturally in tea, coffee, and chocolate, and is commonly added to soft drinks, energy drinks and some foods. However, because of the bitter taste of caffeine, the flavor of drinks or foods having a relatively high caffeine content can be unappealing. Caffeine may include synthetic caffeine and/or natural caffeine, such as coffee bean-extracted caffeine. In at least one example embodiment, the oral pouch product includes caffeine in an amount greater than or equal to about 10 mg (e.g., greater than or equal to about 25 mg, greater than greater than or equal to about 150 mg) The caffeine may be included in an amount less than or equal to about 200 mg (e.g., less than or equal to about 150 mg, less than or equal to about 100 mg, less than or equal to about 75 mg, less than or equal to about 50 mg, or less than or equal to about 25 mg).

The compositions for human consumption may have a relatively high caffeine content so as to provide a consumer with a burst of energy. Moreover, the compositions for human consumption contain about 50 mg to about 200 mg of caffeine or about 75 mg to about 175 mg of caffeine (e.g., 100 mg to about 150 mg of caffeine) so as to provide a burst of energy to the consumer. The composition provides a single serving of a food, drink, oral tobacco product or oral non-tobacco product. A single serving of food can have a weight of about 5 g to about 450 g. A single serving of drink is about 200 mL to about 600 mL. A single serving of an oral pouch product includes one oral pouch product formed as described herein.

Optionally, the composition for human consumption can also include additional energizing ingredients in addition to the caffeine complex. Suitable additional energizing ingredients include, without limitation, taurine, citicoline, and guarana. The additional energizing ingredients can be included in an amount of about 0.1% to about 5% by weight based on the weight of the composition for human consumption.

In at least one example embodiment, the soothing agent includes theanine, melatonin, or both theanine and melatonin. The soothing agent may also include, for example only, chamomile, lavender, jasmine, soursop, cannabidiol, or any combination thereof. The soothing agent can be added as a flavorant and or aroma embedded in the product and/or the package. In at least one example embodiment, the oral pouch product includes the soothing agent in an amount greater than or equal to about 0.1 weight percent (e.g., greater than or equal to about 0.5 weight percent, greater than or equal to about 1 weight percent, greater than or equal to about 1.5 weight percent, greater than or equal to about 2 weight percent, greater than or equal to about 2.5 weight percent, greater than or equal to about 3 weight percent, greater than or equal to about 3.5 weight percent, greater than or equal to about 4 weight percent, or greater than or equal to about 4.5 weight percent). In at least one example embodiment, the oral pouch product includes the soothing agent in an amount less than or equal to about 5 weight percent (e.g., less than or equal to about 4.5 weight percent, less than or equal to about 4 weight percent, less than or equal to about 3.5 weight percent, less than or equal to about 3 weight percent, less than or equal to about 2.5 weight percent, less than or equal to about 2 weight percent, less than or equal to about 1.5 weight percent, less than or equal to about 1 weight percent, or less than or equal to about 0.5 weight percent).

In at least one example embodiment, the oral pouch product includes a plasticizer. The plasticizer may include, for example, a monoglyceride, a diglyceride, a triglyceride (e.g., long, medium, and/or short chain), triacetin, propylene glycol, glycerin, vegetable oil, a phthalate, an ester of a polycarboxylic acid with a linear or branched aliphatic alcohol of moderate chain length, or any combination thereof. In at least one example embodiment, the plasticizer may be present in addition to triglycerides and/or other oils in the liquid mixture.

In at least one example embodiment, the oral pouch product includes a mouth-soluble fiber. In at least one example embodiment, the mouth-soluble fibers include maltodextrin, psyllium, starch, or any combination thereof. In at least one example embodiment, the mouth-soluble fibers include soluble dietary fibers. In at least one example embodiment oral pouch product includes partially-soluble fibers, such as sugar beet fibers.

In at least one example embodiment, the oral pouch product includes minerals in addition to any that may be present due to the inclusion, for example, of fruits and/or vegetables. The minerals may include, for example, calcium, magnesium, phosphorus, iron, zinc, iodine, selenium, potassium, copper, manganese, molybdenum, chromium, and any combination thereof. The amount of minerals incorporated into the oral pouch product for adult human consumption can be varied according to the type of mineral and the intended adult consumer. The amount of minerals may be formulated to include an amount less than or equal to the recommendations of the United States Department of Agriculture Recommended Daily Allowances.

In at least one example embodiment, the focusing agent includes Ginkgo biloba.

The at least one sensate or chemesthesis agent may include mint, menthol, cinnamon, pepper, jambu, or any combination thereof. The at least one sensate or chemesthesis agent may include any soothing, cooling, and/or warming agent. For example, in some example embodiments, the at least one sensate or chemesthesis agent may include capsaicin, pipeline, alpha-hydroxy-sanshool, and (8)-gingerole, which may be selected so as to provide a warm, tingling or burning sensation. In other example embodiments, the at least one sensate or chemesthesis agent may include menthol, menthyl lactate, WS-3 (N-Ethyl-p menthane-3-carboxamide), WS-23 (2-Isopropyl-N,2,3-trimethylbutyramide) and Evercool 180™ (available from Givaudan SA), which may be selected so as to provide a cooling sensation. The at least one sensate or chemesthesis agent may be included in an amount ranging from about 0.01% by weight to about 5% by weight based on the weight of the oral pouch product.

The antioxidant may include, for example, vitamin C, vitamin B, magnesium, calcium, or any combination thereof. In at least one example embodiment, the oral pouch product may include one or more vitamins in addition to any that may be present due to the inclusion, for example, of fruits and/or vegetables. The one or more additional vitamins may include, for example, vitamin A (retinol), vitamin D (cholecalciferol), vitamin E group, vitamin K group (phylloquinones and menaquinones), thiamine (vitamin B1), riboflavin (vitamin B2), niacin, niacinamide, pyridoxine (vitamin B6 group), folic acid, choline, inositol, vitamin B12 (cobalamins), PABA (para aminobezoic acid), biotin, vitamin C (ascorbic acid), and any combination thereof. The amount of vitamins may be chosen so as to provide an amount less than or equal to the recommendations of the United States Department of Agriculture Recommended Daily Allowances.

Suitable minerals include, without limitation, calcium, magnesium, phosphorus, iron, zinc, iodine, selenium, potassium, copper, manganese, molybdenum, chromium, and mixtures thereof. The amount of minerals incorporated into the composition for human consumption can be varied according to the type of mineral and the intended adult consumer. For example, the amount of minerals may be formulated to include an amount less than or equal to the recommendations of the United States Department of Agriculture Recommended Daily Allowances.

Amino acids can also be included in the composition for human consumption. Suitable amino acids include, without limitation, the eight essential amino acids that cannot be biosynthetically produced in humans, including valine, leucine, isoleucine, lysine, threonine, tryptophan, methionine, and phenylalanine. Examples of suitable amino acids include the non-essential amino acids including alanine, arginine, asparagine, aspartic acid, cysteine, glutamic acid, glutamine, glycine, histidine, proline, serine, and tyrosine. The amino acids can be included in an amount of about 0.1% to about 5% by weight based on the weight of the composition.

In at least one example embodiment, the oral pouch product includes a coloring agent. The coloring agent may include a natural colorant, an artificial colorant, or any combination thereof.

In at least one example embodiment, the oral pouch product includes the amino acid in an amount greater than or equal to about 0.1 weight percent (e.g., greater than or equal to about 0.5 weight percent, greater than or equal to about 1 weight percent, greater than or equal to about 1.5 weight percent, greater than or equal to about 2 weight percent, greater than or equal to about 2.5 weight percent, greater than or equal to about 3 weight percent, greater than or equal to about 3.5 weight percent, greater than or equal to about 4 weight percent, or greater than or equal to about 4.5 weight percent). In at least one example embodiment, the oral pouch product includes the amino acid in an amount less than or equal to about 5 weight percent (e.g., less than or equal to about 4.5 weight percent, less than or equal to about 4 weight percent, less than or equal to about 3.5 weight percent, less than or equal to about 3 weight percent, less than or equal to about 2.5 weight percent, less than or equal to about 2 weight percent, less than or equal to about 1.5 weight percent, less than or equal to about 1 weight percent, or less than or equal to about 0.5 weight percent).

In at least one example embodiment, the oral pouch product is free of a pH modifier. In at least one example embodiment, the oral pouch product includes a pH modifier. The pH modifier may include, for example, ammonium carbonate, ammonium bicarbonate, ammonium hydroxide, calcium carbonate, potassium carbonate, potassium bicarbonate, potassium hydroxide, sodium carbonate, sodium bicarbonate, sodium hydroxide, or any combination thereof.

In at least one example embodiment, the oral pouch product includes the pH adjuster in an amount less than or equal to about 2 weight percent (e.g., less than or equal to about 1 weight percent, less than or equal to about 0.5 weight percent, less than or equal to about 0.1 weight percent, or less than or equal to about 0.05 weight percent). In at least one example embodiment, the oral pouch product includes the pH adjuster in an amount ranging from about 0.01 weight percent to 2 weight percent.

In at least one example embodiment, the oral pouch product is free of a wax. In at least one example embodiment, the oral pouch product includes a wax. The wax may include, for example, paraffin, microcrystalline wax, or both paraffin and microcrystalline wax.

In at least one example embodiment, the oral pouch product is free of a filler. In at least one example embodiment, the oral pouch product includes a filler. The filler may be configured to alter a texture or pliability of the oral pouch product. The filler may include mouth-soluble elements, mouth-insoluble elements, or both mouth-soluble and mouth-insoluble elements. Mouth-soluble elements may be configured to dissolve or disintegrate when in an adult consumer's mouth so as to render the oral pouch product more pliable. The filler may include, for example, dicalcium phosphate, calcium sulfate, a clay, silica, glass particles, glyceryl palmitostearate, sodium stearyl fumarate, talc, or any combination thereof. In at least one example embodiment, certain other compounds or elements or components, including, for example, mouth-soluble fibers, sweeteners, minerals, as described, may be classified as fillers. For example, in at least one example embodiment, cellulosic materials may be present in the oral pouch product as fillers in addition to or as an alternative to being carriers for the liquid mixture.

In at least one example embodiment, the oral pouch product includes the filler in an amount less than or equal to 20 weight percent (e.g., less than or equal to 15 weight percent, less than or equal to 10 weight percent, less than or equal to 9 weight percent, less than or equal to 8 weight percent, less than or equal to 7 weight percent, less than or equal to 6 weight percent, less than or equal to 5 weight percent, less than or equal to 4 weight percent, less than or equal to 3 weight percent, less than or equal to 2 weight percent, or less than or equal to 1 weight percent). In at least one example embodiment, the oral pouch product includes the filler in an amount greater than or equal to about 0 weight percent (e.g., greater than or equal to about 1 weight percent, greater than or equal to about 2 weight percent, greater than or equal to about 3 weight percent, greater than or equal to about 4 weight percent, or greater than or equal to about 5 weight percent). In at least one example embodiment, the oral pouch product includes the filler in an amount ranging from about 0 weight percent to about 8 weight percent.

In at least one example embodiment, the oral pouch product is free of a flavorant. In at least one example embodiment, the oral pouch product includes a flavorant. In at least one example embodiment, the oral pouch product includes an encapsulated flavorant. The flavorant may be natural or artificial. The flavorant may include, for example, a fruit flavorant (e.g., bergamot, berry, cherry, lemon, and/or orange), a liquor or liqueur flavorant (e.g., bourbon, cognac, scotch, whiskey, and/or DRAMBUIE brand liqueur), a mint flavorant (e.g., Japanese mint, menthol, peppermint, spearmint, wintergreen, and/or mint oils from a species of the genus Mentha), a floral flavorant (e.g., geranium, lavender, and/or rose), a spice, an herb, or another botanical or botanical-derived flavorant (e.g., anise, apium graveolens, caraway, cardamom, cascarilla, cassia, cinnamon, chamomile, clove, cocoa, coffee, coriander, fennel, ginger, jasmine, licorice, nutmeg, pimenta, sage, sandalwood vanilla, and/or ylang-ylang), honey essence, or any combination thereof. In at least one example embodiment, the flavorant includes bergamot, berry, cherry, lemon, orange, bourbon, cognac, scotch, whiskey, DRAMBUIE brand liqueur, Japanese mint, menthol, peppermint, spearmint, wintergreen, mint oils from a species of the genus Mentha, geranium, lavender, rose, anise, apium graveolens, caraway, cardamom, cascarilla, cassia, cinnamon, chamomile, clove, coffee, coriander, fennel, ginger, jasmine, licorice, nutmeg, pimenta, sage, sandalwood vanilla, ylang-ylang, honey essence, or any combination thereof.

In at least one example embodiment, the oral pouch product is free of a carrier. In at least one example embodiment, the oral pouch product includes a carrier. The carrier may include a liquid carrier. The liquid carrier may include, for example, water, propylene, glycol, glycerin, ethanol, or any combination thereof.

In at least one example embodiment, the oral pouch product is free of an oil. In at least one example embodiment, the oral pouch product includes an oil. The oil may include, for example, a monoglyceride, a diglyceride, a triglyceride, triacetin, a flavor oil, or any combination thereof. The flavor oil may be or may include a flavorant.

An amount of oil may be selected to achieve a desired texture and/or softness. For example, the softness of the oral pouch product may be increased as an amount of the oil in the oral pouch product increases. In at least one example embodiment, the oral pouch product includes oil in an amount greater than or equal to about 5 weight percent (e.g., greater than or equal to about 8 weight percent, greater than or equal to about 9 weight percent, greater than or equal to about 10 weight percent, greater than or equal to about 11 weight percent, greater than or equal to about 12 weight percent, greater than or equal to about 13 weight percent, or greater than or equal to about 15 weight percent). In at least one example embodiment, the oral pouch product includes oil in an amount less than or equal to about 20 weight percent (e.g., less than or equal to about 15 weight percent, less than or equal to about 14 weight percent, less than or equal to about 13 weight percent, less than or equal to about 12 weight percent, less than or equal to about 11 weight percent, less than or equal to about 10 weight percent, or less than or equal to about 8 weight percent). In at least one example embodiment, the oral pouch product includes the oil in an amount ranging from about 8 weight percent to about 16 weight percent (e.g., about 10 weight percent to about 14 weight percent, about 11 weight percent to about 13 weight percent, or about 12 weight percent).

The oral pouch product (e.g., the oral tobacco product, the oral non-tobacco product, or the oral cannabis product) may have various ranges of moisture. In at least one example embodiment, the oral pouch product has a moisture content ranging from 1% by weight to 30% by weight. In at least one example embodiment, the oral pouch product has a medium moisture content, such as a moisture content ranging from 10% by weight to 30% by weight. In at least one example embodiment, the oral pouch product is a wet oral pouch product having a moisture content ranging from 20% by weight to 30% by weight.

### Oral Pouch Product

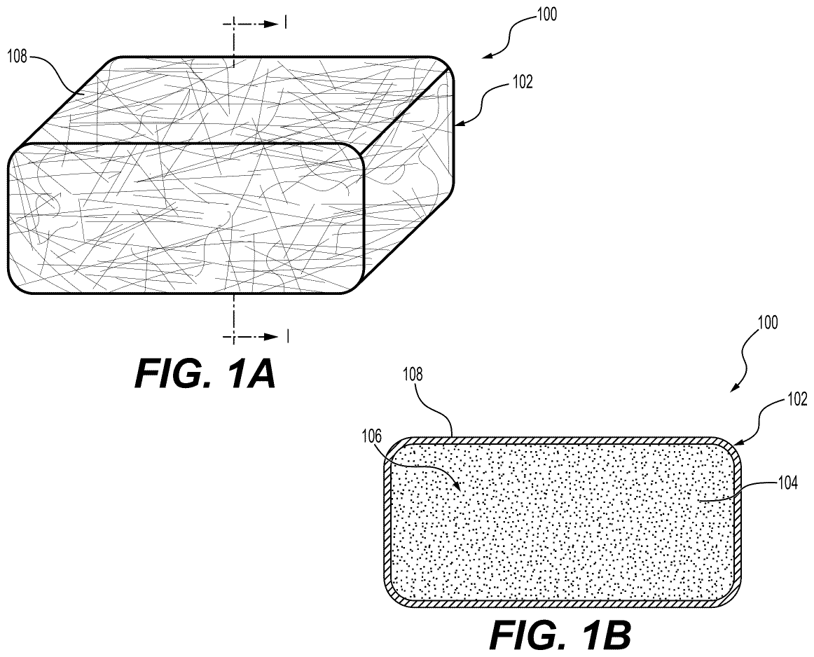

FIG. 1A is a perspective view of an oral pouch product according to at least one example embodiment.

FIG. 1B is a cross sectional view of the oral pouch product taken at line I-I of FIG. 1A according to at least one example embodiment.

In at least one example embodiment, as shown in FIGS. 1A-1, an oral pouch product 100 is configured to fit in an adult consumer's mouth. The oral pouch product 100 includes a pouch wrapper 102 and an inner filler 104. The filler 104 is in an inner cavity 106 at least partially defined by the pouch wrapper 102. For example, an adult consumer can suck, chew, or otherwise orally manipulate the oral pouch product 100 to release flavor and/or functional ingredients contained therein.

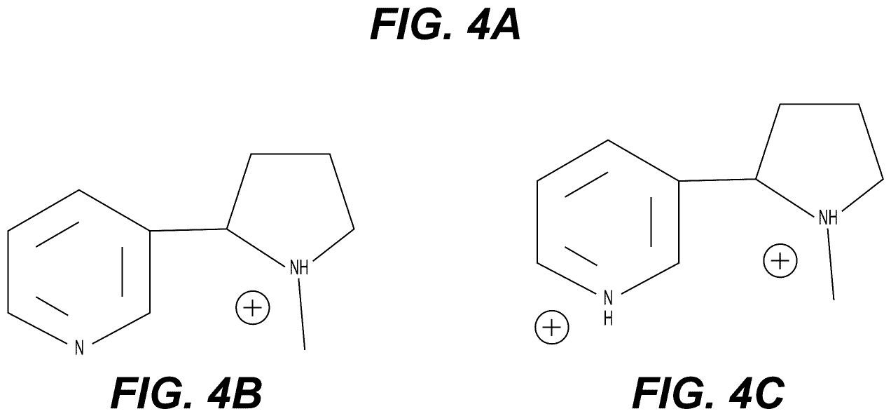

The filler 104 includes a dry mixture and a liquid mixture, such as the liquid mixture described herein with respect to FIGS. 4A-7D. At least a portion of the liquid mixture is absorbed on the dry mixture. In at least one example embodiment, all of the liquid mixture is absorbed on the dry mixture. The liquid mixture includes an oil and an active pharmaceutical ingredient (API), such as nicotine. The dry mixture includes an insoluble material or carrier, such as a cellulosic material.

In at least one example embodiment, the API includes nicotine and the oral pouch product 100 includes oil, water, and the nicotine in the amounts and ratios described below with respect to FIGS. 4A-7D. In at least one example embodiment, the API includes one or more tobacco extracts, nicotine, one or more cannabis extracts, and/or one or more cannabinoids. The nicotine may include tobacco-derived nicotine and/or synthetic nicotine. In at least one example embodiment, the API includes liquid nicotine. The cannabinoid may include tetrahydrocannabinol (“THC”) and/or cannabidiol (“CBD”).

The oral pouch product 100 may include the API in an amount greater than or equal to about 0.1 weight percent (e.g., greater than or equal to about 0.5 weight percent, greater than or equal to about 1 weight percent, greater than or equal to about 2 weight percent, greater than or equal to about 3 weight percent, greater than or equal to about 4 weight percent, greater than or equal to about 5 weight percent, greater than or equal to about 6 weight percent, greater than or equal to about 7 weight percent, greater than or equal to about 8 weight percent, or greater than or equal to about 9 weight percent). The oral pouch product 100 may include the API in an amount less than or equal to about 10 weight percent (e.g., less than or equal to about 9 weight percent, less than or equal to about 8 weight percent, less than or equal to about 7 weight percent, less than or equal to about 6 weight percent, less than or equal to about 5 weight percent, less than or equal to about 4 weight percent, less than or equal to about 3 weight percent, less than or equal to about 2 weight percent, less than or equal to about 1 weight percent, or less than or equal to about 0.5 weight percent).

In at least one example embodiment, the oil of the liquid mixture includes one or more triglycerides, one or more diglycerides, one or more monoglycerides, triacetin, triolein, trilinolein, vegetable oil, one or more partially-hydrogenated oils, or any combination thereof. In at least one example embodiment, the oil consists essentially of one or more triglycerides, one or more diglycerides, one or more monoglycerides, or any combination thereof. In at least one example embodiment, the oil consists essentially of the triglyceride. The triglyceride may include one or more short-chain triglycerides (“SCT”), one or more medium chain triglycerides (“MCT”), one or more long chain triglycerides (“LCT”), or any combination thereof. In at least one example embodiment, the triglyceride consists essentially of MCT.

In at least one example embodiment, the filler 104 includes the oil in an amount greater than or equal to about 0.5 weight percent (e.g., greater than or equal to about 1 weight percent, greater than or equal to about 5 weight percent, greater than or equal to about 10 weight percent, greater than or equal to about 15 weight percent, greater than or equal to about 20 weight percent, greater than or equal to about 25 weight percent, greater than or equal to about 30 weight percent, greater than or equal to about 35 weight percent, greater than or equal to about 40 weight percent, or greater than or equal to about 45 weight percent). In at least one example embodiment, the oral pouch product 100 includes the oil in an amount greater than 20 weight percent based on the weight of the pouch. The filler 104 may include the oil in an amount less than or equal to about 50 weight percent (e.g., less than or equal to about 45 weight percent, less than or equal to about 40 weight percent, less than or equal to about 35 weight percent, less than or equal to about 30 weight percent, less than or equal to about 25 weight percent, less than or equal to about 20 weight percent, less than or equal to about 15 weight percent, less than or equal to about 10 weight percent, less than or equal to about 5 weight percent, or less than or equal to about 1 weight percent).

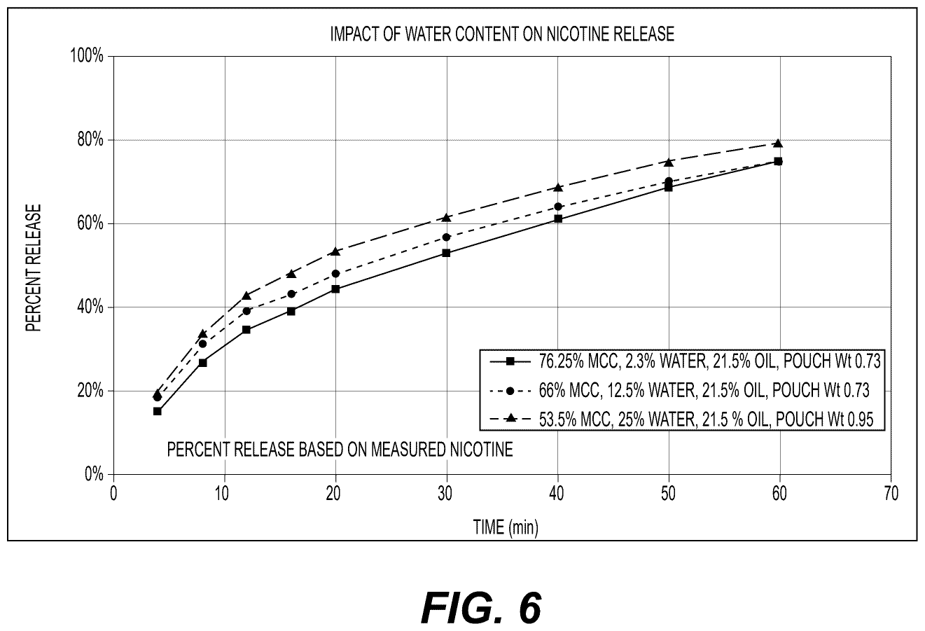

In at least one example embodiment, the filler 104 includes water in amount less than or equal to about 35 weight percent (e.g., less than or equal to about 30 weight percent, less than or equal to about 25 weight percent, less than or equal to about 20 weight percent, less than or equal to about 20 weight percent, or less than or equal to about 15 weight percent). In at least one example embodiment, the filler 104 includes water in an amount greater than or equal to about 1 weight percent (e.g., greater than or equal to about 5 weight percent, greater than or equal to about 10 weight percent, greater than or equal to about 15 weight percent, greater than equal to about 20 weight percent, greater than or equal to about 25 weight percent, or greater than equal to about 30 weight percent). In at least one example embodiment, by including water in the liquid mixture, for example between about 10 weight percent and about 30 weight percent, the release rate of nicotine to a user may be increased as described in more detail with regard to FIG. 6.

In at least one example embodiment, a weight ratio of the dry mixture to the liquid mixture may be greater than or equal to about 50:50 (e.g., greater than or equal to about 60:40, greater than or equal to about 65:35, greater than or equal to about 70:30, greater than or equal to about 75:25, greater than or equal to about 80:20, greater than or equal to about 85:15, greater than or equal to about 90:10, or greater than or equal to about 95:5).

In at least one example embodiment, the dry mixture includes an insoluble material. At least a portion of the liquid mixture may be absorbed in the insoluble material, as described below with respect to FIGS. 1A-3D. In at least one example embodiment, all of the liquid mixture is absorbed in the insoluble material.

In at least one example embodiment, the insoluble material is a cellulosic material. The cellulosic material may include or be derived from sugar beets, wood pulp, cotton, bran, citrus pulp, grass (e.g., switch grass), willow, poplar, or any combination thereof. The insoluble cellulosic material may be a treated cellulosic material, such as microcrystalline cellulose (“MCC”), carboxymethyl cellulose (“CMC”), hydroxypropyl methylcellulose (“HPMC”), hydroxypropyl cellulose (“HPC”), or any combination thereof. In at least one example embodiment, the cellulosic material includes mouth-insoluble cellulosic fibers. In at least one example embodiment, the cellulosic material includes MCC. In at least one example embodiment, the MCC includes wood pulp-derived MCC.

In at least one example embodiment, the insoluble material includes water-insoluble fibers. The water-insoluble fibers may include water-insoluble polymeric fibers. The water-insoluble polymer fibers may include polypropylene, polyester, polyethylene, polyurethane, or any combination thereof.

In at least one example embodiment, the filler 104 includes the insoluble material in an amount greater than or equal to about 10 weight percent (e.g., greater than or equal to about 15 weight percent, greater than or equal to about 20 weight percent, greater than or equal to about 25 weight percent, greater than or equal to about 30 weight percent, greater than or equal to about 35 weight percent, greater than or equal to about 40 weight percent, greater than or equal to about 45 weight percent, greater than or equal to about 50 weight percent, greater than or equal to about 55 weight percent, greater than or equal to about 60 weight percent, greater than or equal to about 65 weight percent, greater than or equal to about 70 weight percent, or greater than or equal to about 75 weight percent). The filler 104 may include the insoluble material in an amount less than or equal to about 80 weight percent (e.g., less than or equal to about 75 weight percent, less than or equal to about 70 weight percent, less than or equal to about 65 weight percent, less than or equal to about 60 weight percent, less than or equal to about 55 weight percent, less than or equal to about 50 weight percent, less than or equal to about 45 weight percent, less than or equal to about 40 weight percent, less than or equal to about 35 weight percent, less than or equal to about 30 weight percent, less than or equal to about 25 weight percent, less than or equal to about 20 weight percent, or less than or equal to about 15 weight percent). In at least one example embodiment, the filler 104 includes the insoluble material in an amount ranging from 10 weight percent to 70 weight percent (e.g., about 30 weight percent to about 60 weight percent).

In at least one example embodiment, the insoluble material has a particle size of greater than or equal to about 50 microns (e.g., greater than or equal to about 100 microns, greater than or equal to about 150 microns, greater than or equal to about 200 microns, greater than or equal to about 250 microns, greater than or equal to about 300 microns, greater than or equal to about 350 microns, greater than or equal to about 400 microns, or greater than or equal to about 450 microns). The particle size may be less than or equal to about 500 microns (e.g., less than or equal to about 450 microns, less than or equal to about 400 microns, less than or equal to about 350 microns, less than or equal to about 300 microns, less than or equal to about 250 microns, less than or equal to about 200 microns, less than or equal to about 150 microns, or less than or equal to about 100 microns).

In at least one example embodiment, the dry mixture further includes a water-soluble filler. The water-soluble filler may include one or more sweeteners, maltodextrin, psyllium, starch, one or more polysaccharides, one or more salts, one or more synthetic polymers, dietary fibers, or any combination thereof. The filler 104 may include the water-soluble filler in an amount greater than or equal to about 10 weight percent (e.g., greater than or equal to about 15 weight percent, greater than or equal to about 20 weight percent, greater than or equal to about 25 weight percent, greater than or equal to about 30 weight percent, greater than or equal to about 35 weight percent, greater than or equal to about 40 weight percent, or greater than or equal to about 45 weight percent). The filler 104 may include the water-soluble filler in an amount less than or equal to about 50 weight percent (e.g., less than or equal to about 45 weight percent, less than or equal to about 40 weight percent, less than or equal to about 35 weight percent, less than or equal to about 30 weight percent, less than or equal to about 25 weight percent, less than or equal to about 20 weight percent, or less than or equal to about 15 weight percent).

In at least one example embodiment, the filler 104 includes at least one sweetener as described above. In at least one example embodiment, the sweetener includes one or more sugar alcohols as described above. The filler 104 may include the sugar alcohol in an amount greater than or equal to about 10 weight percent (e.g., greater than or equal to about 15 weight percent, greater than or equal to about 20 weight percent, greater than or equal to about 25 weight percent, greater than or equal to about 30 weight percent, greater than or equal to about 35 weight percent, greater than or equal to about 40 weight percent, or greater than or equal to about 45 weight percent). The filler 104 may include the sugar alcohols in an amount less than or equal to about 50 weight percent (e.g., less than or equal to about 45 weight percent, less than or equal to about 40 weight percent, less than or equal to about 35 weight percent, less than or equal to about 30 weight percent, less than or equal to about 25 weight percent, less than or equal to about 20 weight percent, or less than or equal to about 15 weight percent). In at least one example embodiment, the filler 104 includes the sugar alcohol in an amount ranging from about 15 weight percent to about 25 weight percent (e.g., about 18 weight percent to about 22 weight percent).

In at least one example embodiment, the filler 104 includes a pH adjuster as described above. The pH adjuster may be included in an amount greater than or equal to about 0.01 weight percent (e.g., greater than or equal to about 0.05 weight percent, greater than or equal to about 0.1 weight percent, greater than or equal to about 0.5 weight percent, greater than or equal to about 1 weight percent, greater than or equal to about 2 weight percent, greater than or equal to about 3 weight percent, greater than or equal to about 4 weight percent, greater than or equal to about 5 weight percent, greater than or equal to about 6 weight percent, greater than or equal to about 7 weight percent, greater than or equal to about 8 weight percent, or greater than or equal to about 9 weight percent). The pH adjuster may be included in an amount less than or equal to about 2 weight percent (e.g., less than or equal to about 10 weight percent, less than or equal to about 9 weight percent, less than or equal to about 8 weight percent, less than or equal to about 7 weight percent, less than or equal to about 6 weight percent, less than or equal to about 5 weight percent, less than or equal to about 4 weight percent, less than or equal to about 3 weight percent, less than or equal to about 2 weight percent, less than or equal to about 1 weight percent, less than or equal to about 0.5 weight percent, less than or equal to about 0.1 weight percent, or less than or equal to about 0.05 weight percent). In at least one example embodiment, the pH adjuster is present in an amount ranging from about 4 weight percent to about 6 weight percent. In at least one other example embodiment, the pH adjuster is present in an amount ranging from about 0.01 weight percent to about 2 weight percent. In at least one other example embodiment, the oral pouch product is free of a pH adjuster.

In at least one example embodiment, the oral pouch product is free of humectants. In at least one example embodiment, the oral pouch product is free of glycerin and/or propylene glycol.

In at least one example embodiment, the filler 104 includes an antioxidant as described above. The filler 104 may include the antioxidant in an amount less than or equal to about 5 weight percent (e.g., less than or equal to about 4 weight percent, less than or equal to about 3 weight percent, less than or equal to about 2 weight percent, less than or equal to about 1 weight percent, or less than or equal to about 0.5 weight percent).

In at least one example embodiment, the filler 104 includes one or more flavorants as described above. In at least one example embodiment, the oral pouch product includes an encapsulated flavorant. In at least one example embodiment, the filler 104 includes the flavorant in an amount greater than or equal to about 0.01 weight percent (e.g., greater than or equal to about 0.1 weight percent, greater than or equal to about 0.5 weight percent, greater than or equal to about 1 weight percent, greater than or equal to about 2 weight percent, greater than or equal to about 4 weight percent, greater than or equal to about 5 weight percent, greater than or equal to about 8 weight percent, greater than or equal to about 10 weight percent, greater than or equal to about 15 weight percent, or greater than or equal to about 20 weight percent). The filler 104 may include the one or flavorants in an amount less than or equal to about 25 weight percent (e.g., less than or equal to about 20 weight percent, less than or equal to about 15 weight percent, less than or equal to about 10 weight percent, greater than or equal to about 8 weight percent, less than or equal to about 5 weight percent, less than or equal to about 4 weight percent, greater than or equal to about 2 weight percent, less than or equal to about 1 weight percent, or less than or equal to about 0.5 weight percent, less than or equal to about 0.1 weight percent). In at least one example embodiment, the filler 104 includes the flavorant in an amount ranging from about 0.1 weight percent to about 10 weight percent (e.g., about 0.1 weight percent to about 4 weight percent).

In at least one example embodiment, the filler 104 includes an emulsifier and/or surfactant. The emulsifier and/or surfactant may include lecithin, glycerol monosterate, a polysorbate, a poloxamer, or a combination of lecithin and glycerol monostearate. The emulsifier and/or surfactant may be present in an amount greater than 0 weight percent (e.g., greater than or equal to about 0.5 weight percent, greater than or equal to about 0.75 weight percent, greater than or equal to about 1 weight percent, greater than or equal to about 2 weight percent, greater than or equal to about 3 weight percent, or greater than or equal to about 4 weight percent). The emulsifier and/or surfactant may be present in an amount less than or equal to about 5 weight percent (e.g., less than or equal to about 4 weight percent, less than or equal to about 3 weight percent, less than or equal to about 2 weight percent, less than or equal to about 1 weight percent, less than or equal to about 0.75 weight percent, or less than or equal to about 0.5 weight percent). In at least one example embodiment, the emulsifier and/or surfactant is present in an amount ranging from 0.1 weight percent to about 2 weight percent.

In at least one example embodiment, the dry mixture may additionally or alternatively include cannabis. The cannabis can include any portion of the cannabis plant and/or any extract therefrom as described above.

In at least one example embodiment, prior to placement of the cannabis (or other plant material) in the oral pouch product 100, the cannabis (or other plant material) may be heated to a temperature sufficient to decarboxylate a compound within the cannabis (or other plant material). For example, cannabis may be maintained at the heated temperature for a time period that is sufficient to cause decarboxylation (i.e., to convert tetrahydrocannabinolic acid (“THCA”) that is present in the cannabis to THC, and/or to convert cannabidiolic acid (“CBDA”) to CBD). For some applications, maintaining the plant material at the heated temperature causes the decarboxylation of the cannabis in accordance with an article by Dussy, et al., entitled “Isolation of Delta9-THCA-A from hemp and analytical aspects concerning the determination of Delta9-THC in cannabis products (Forensic Sci. Int. 2005 Apr. 20; 149(1):3-10), the entire disclosure of which is incorporated herein by reference, and/or an article by Veress, et al., entitled “Determination of cannabinoid acids by high-performance liquid chromatography of their neutral derivatives formed by thermal decarboxylation: I. Study of the decarboxylation process in open reactors” (Journal of Chromatography A 520:339-347, November 1990), the entire disclosure of which is incorporated herein by reference. For example, the cannabis (or other plant material) may be maintained at approximately 225 degrees Celsius for approximately 35 to 45 minutes.

In at least one example embodiment, the dry mixture can also include tobacco material (in addition to or in lieu of the cannabis material and/or cellulose) as described above. In at least one other example embodiment, the filler 104 includes tobacco plant material in an amount less than or equal to about 5 weight percent (e.g., less than or equal to about 4 weight percent, less than or equal to about 3 weight percent, less than or equal to about 2 weight percent, or less than or equal to about 1 weight percent). In at least one other example embodiment, the filler 104 is free of tobacco plant material.

In at least one example embodiment, the filler 104 includes one or more additives as described above. The oral pouch product may include multiple additional elements. Additionally, a single element may belong to more than one of the categories above. In at least one example embodiment, the filler 104 includes the one or more additives in an amount greater than or equal to about 0.01 weight percent (e.g., greater than or equal to about 0.5 weight percent, greater than or equal to about 1 weight percent, greater than or equal to about 5 weight percent, greater than or equal to about 10 weight percent, greater than or equal to about 15 weight percent, greater than or equal to about 20 weight percent, or greater than or equal to about 25 weight percent). The filler 104 may include the one or more additives in an amount less than or equal to about 30 weight percent (e.g., less than or equal to about 25 weight percent, less than or equal to about 20 weight percent, less than or equal to about 15 weight percent, less than or equal to about 10 weight percent, less than or equal to about 5 weight percent, less than or equal to about 1 weight percent, or less than or equal to about 0.5 weight percent).

In at least one embodiment, the one or more materials disposed in the inner cavity 106 as the filler 104, including, for example only, the one or more additives and/or one or more functional ingredients, may be provided in the form of a plurality of capsules, microcapsules, and/or beads of various sizes. The capsules, microcapsules, and/or beads may have a size that is determined by the desired size of the final product (e.g., oral pouch product 400). For example, the capsules, microcapsules, and/or beads may range in size from about 0.1 mm to about 8 mm depending on the ingredients contained therein.

In each instance, each capsule, microcapsule, and/or bead may include an outer shell and an inner core. Varying the thicknesses of the outer shells of the capsules, microcapsules, and/or beads included can allow for the ingredients contained in each of the capsules, microcapsules, and/or beads to be released at varying rates to prolong the flavor and/or functional experience of the oral pouch product 100. In some example embodiments, the shells range in thickness from about 0.1 mm to about 7 mm, depending on the size of the capsules, microcapsules, and/or beads and a desired dissolution rate. The capsules, microcapsules, and/or beads having the thinnest shells dissolve first to release the enclosed flavors and functional ingredients. Capsules, microcapsules, and/or beads having thicker shells dissolve at a slower rate to provide continued flavor and functional ingredients.

In at least one example embodiment, one or more materials in the filler 104 may be provided as granules, such as encapsulated nicotine granules and/or encapsulated sweetener granules, as described in U.S. patent application Ser. No. 17/223,773, the entire contents of which is incorporated herein by reference.

In at least one example embodiment, the pouch wrapper 102 includes an outer web 108. The outer web 108 may be formed from a material that is generally recognized as safe (“GRAS”) for use and/or contact with food. The material may be stain resistant, water permeable, and/or porous. The outer web 108 may have a thickness of about 0.05 mm to about 0.25 mm (e.g., about 0.075 mm to about 0.100 mm or about 0.1 mm to about 0.20 mm). The outer web 108 can be formed of a permeable material or a semi-permeable material, such that, for example, saliva, water, or both saliva and water can pass through the outer web 108 and into the inner cavity 106 defined by the pouch wrapper 102. Flavors and juices formed by mixing saliva and/or water with the filler 104 contained within the oral pouch product 100 can be drawn out of the oral pouch product 100 through the outer web 108.

In at least one example embodiment, the wrapper 102 includes a non-woven material. The non-woven material may be formed of a polymer, including one or more synthetic and/or natural polymers. For example, wrapper 102 may be formed of a mesh material formed of spun or melt-blown fibers, such as polyurethane fibers as described in U.S. Pat. Nos. 10,448,669, 10,463,070, and/or 9,414,624, the entire contents of each of which is incorporated herein by reference thereto. The mesh material may be at least partially elastomeric. The mesh material may be white and may exclude any colorant. In other example embodiments, the mesh material may include a colorant. Further, because of the material used to form the wrapper 102, the oral pouch product 100 may exclude seams so as to provide a softer pouch.

In at least one example embodiment, the mesh material has a basis weight of greater than or equal to about 10 grams per square meter (“gsm”) (e.g., greater than or equal to about 15 gsm, greater than or equal to about 20 gsm, greater than or equal to about 25 gsm, greater than or equal to about 30 gsm, greater than or equal to about 35 gsm, greater than or equal to about 40 gsm, or greater than or equal to about 45 gsm). The basis weight may be less than or equal to about 50 gsm (e.g., less than or equal to about 45 gsm, less than or equal to about 40 gsm, less than or equal to about 35 gsm, less than or equal to about 30 gsm, less than or equal to about 25 gsm, less than or equal to about 20 gsm, or less than or equal to about 15 gsm). In at least one example embodiment, the basis weight ranges from about 20 gsm to about 30 gsm.

In at least one other example embodiment, the outer web 108 comprises a paper. For example, the outer web 108 can be formed of a cellulose fiber material, such as tea bag material or other materials typically used to form snus pouches. In at least one example embodiment, the outer web 108 has a desired (or alternatively, predetermined) level for basis weight and/or wet strength to reduce occurrence of breakage of the pouch wrapper 102 during manufacturing operations, storage, and placement in an adult consumer's mouth. For example, the outer web 108 may comprise a tea bag material having a basis weight of about 16.5 gsm with a wet tensile CD strength of 68 N/m. In another example embodiment, the outer web 108 may be formed of a paper having a wet MD tensile strength of about 45 N/mm to about 52 N/mm.

In at least one example embodiment, the outer web 108 is formed of a hydrophobic paper or material. The hydrophobic paper may be formed of a cellulosic material. The hydrophobic paper may be non-woven material and may include any hydrophobic materials. The hydrophobic materials may be synthetic materials and/or semi-synthetic materials. The hydrophobic materials may include viscose, rayon, lyocell, and/or modal fibers. The outer web 108 may be treated to make the outer web 108 hydrophobic. In other example embodiments, the hydrophobic material may be a woven material.

In at least one example embodiment, the wrapper 102 is free of an added coloring agent. In at least one example embodiment, the wrapper 102 is white due to a natural color of the polymer(s) used to form the wrapper. However, in other example embodiments, the wrapper 102 may be colored to indicate a flavor of the filler 104 contained therein. The color of the wrapper 102 may be selected to identify contents thereof. For example, a green pouch wrapper may be used to identify an oral pouch product including a mint flavorant, while a red pouch wrapper may be used to identify an oral pouch product including cinnamon.

The material used to form the outer web 108 may have a neutral or pleasant taste and/or aroma. In at least one example embodiment, the outer web 108 may be impregnated or coated with at least one flavorant, at least one cannabis material, at least one tobacco material, at least one binder, at least one sensate or chemesthesis agent, at least one functional ingredient, at least one salivation inducing ingredient, or any combination thereof to enhance a flavor of the filler 104 contained within the oral pouch product 100. A substantially continuous coating including the at least one flavorant and/or the at least one cannabis material and/or at least one tobacco material and/or the at least one binder and/or the at least one sensate or chemesthesis agent may be coated on outer (exterior facing) surfaces of the outer web 108. In at least one example embodiment, the coating may be formed on only a portion of the outer web 108, such as only along the seams or only on one side of the oral pouch product 100. The coating can provide an initial flavor burst upon placement of the oral pouch product 100 in an oral cavity, while the filler 104 provides a later flavor release to prolong flavor release during placement in an adult consumer's mouth.

The at least one flavorant may be any flavorant disclosed herein for inclusion in the filler 104. In at least one example embodiment, the at least one flavorant may be coated on or impregnated in the outer web 108 in an amount ranging from about 0.01 weight percent to about weight percent based on the weight of the oral pouch product 100 (e.g., about 0.1 weight percent to about 4.5 weight percent, about 1 weight percent to about 4 weight percent, about 1.5 weight percent to about 3.5 weight percent, about 2 weight percent to about 3 weight percent).

In at least one example embodiment, the at least one cannabis or tobacco material may be coated on or impregnated in the outer web 108. The at least one cannabis or tobacco material may include, for example only, a ground cannabis or tobacco material, cannabis or tobacco plant fibers, and/or any extract thereof. The at least one cannabis or tobacco material may be coated on or impregnated in the outer web 108 in an amount ranging from about 0.01 weight percent to about 5 weight percent based on the weight of oral pouch product 100 (e.g., about 0.1 weight percent to about 4.5 weight percent, about 1 weight percent to about 4 weight percent, about 1.5 weight percent to about 3.5 weight percent, or about 2 weight percent to about 3 weight percent).

In at least one example embodiment, the at least one binder may be coated on or impregnated in the outer web 108. The at least one binder is a food grade adhesive, gum, or other binder. For example, in some example embodiments, the at least one binder includes, without limitation, sodium alginate, sugar, agar, guar gum, and the like. The at least one binder may be coated on or impregnated in the outer web in an amount ranging from about 0.01 weight percent to about 5 weight percent based on the weight of the oral pouch product 100 (e.g., about 0.1 weight percent to about 4.5 weight percent, about 1 weight percent to about 4 weight percent, about 1.5 weight percent to about 3.5 weight percent, about 2 weight percent to about 3 weight percent).

The at least one sensate or chemesthesis agent may be any of those discussed above with respect to the filler 104. The at least one sensate or chemesthesis agent may be coated on or impregnated in the outer web 108 in an amount ranging from about 0.01 weight percent to about 5 weight percent based on the weight of the oral pouch product 100 (e.g., about 0.1 weight percent to about 4.5 weight percent, about 1 weight percent to about 4 weight percent, about 1.5 weight percent to about 3.5 weight percent, or about 2 weight percent to about 3 weight percent).

In at least one example embodiment, the at least one functional ingredient may include an antioxidant, a soothing agent, an energizing agent, an effervescent, or any combination thereof, such as those described above in the discussion of the filler 104. In at least one example embodiment, the at least one functional ingredient may be coated on or impregnated in the outer web 108 in an amount ranging from about 0.01 weight percent to about 5 weight percent based on the weight of the oral pouch product 100 (e.g., about 0.1 weight percent to about 4.5 weight percent, about 1 weight percent to about 4 weight percent, about 1.5 weight percent to about 3.5 weight percent, or about 2 weight percent to about 3 weight percent).

The oral pouch product 100 may be an oral pouch product having, for example only, a generally rectangular shape, a ravioli or pillow shape, an oblong shape, or any other suitable shape. Various shapes may be utilized so long as the shapes fit comfortably and discreetly in an adult consumer's mouth. In at least one example embodiment, the oral pouch product 100 is substantially free of oral cavity irritant, which, as used herein, means that the shape, configuration, and position of the oral pouch product 100 do not irritate oral tissues (e.g., gums) via sharp edges and the like. Furthermore, “substantial” and “substantially free” as used in connection with oral cavity irritant means that the shape, configuration, and position of the oral pouch product 100 does not irritate oral tissues (e.g., gums) in a time frame or period having the same order of magnitude as a typical length of time during which the oral pouch product 100 may be enjoyed by an adult consumer. Generally, sharp corners are avoided as sharp corners may lead to oral discomfort.

The oral pouch product 100 may be sized and configured to fit comfortably in an adult consumer's mouth, such as between the cheek and gum. In at least one example embodiment, the oral pouch product 100 has a major dimension in the range of about 0.20 inch to about 2.0 inches (e.g., about 0.25 inch to 1.75 inches, about 0.75 inch to about 1.5 inch) and a transverse dimension in the range of about 0.25 to about 1.5 inches (e.g., about 0.50 inch to 1.25 inches, about 0.75 inch to about 1.0 inch). The oral pouch product 100 may weigh about 0.25 gram (g) to about 2.0 grams (e.g., about 0.3 gram to about 1.8 grams, about 0.4 gram to about 1.5 grams, about 0.5 grams to about 1.25 grams, or about 0.75 gram to about 1.0 gram).

The oral pouch product 100 may be placed in an adult consumer's mouth for about 1 minute to about 2 hours (e.g., about 1 minute to about 100 minutes, about 5 minutes to about 90 minutes, about 10 minutes to about 80 minutes, about 20 minutes to about 60 minutes). The size of the oral pouch product 100 may be selected based on desired length of placement in an adult consumer's mouth. For example, a larger pouch including a larger amount of filler may provide for longer placement. In at least one example embodiment, the oral pouch product 100 is discarded after a single placement in an adult consumer's mouth.

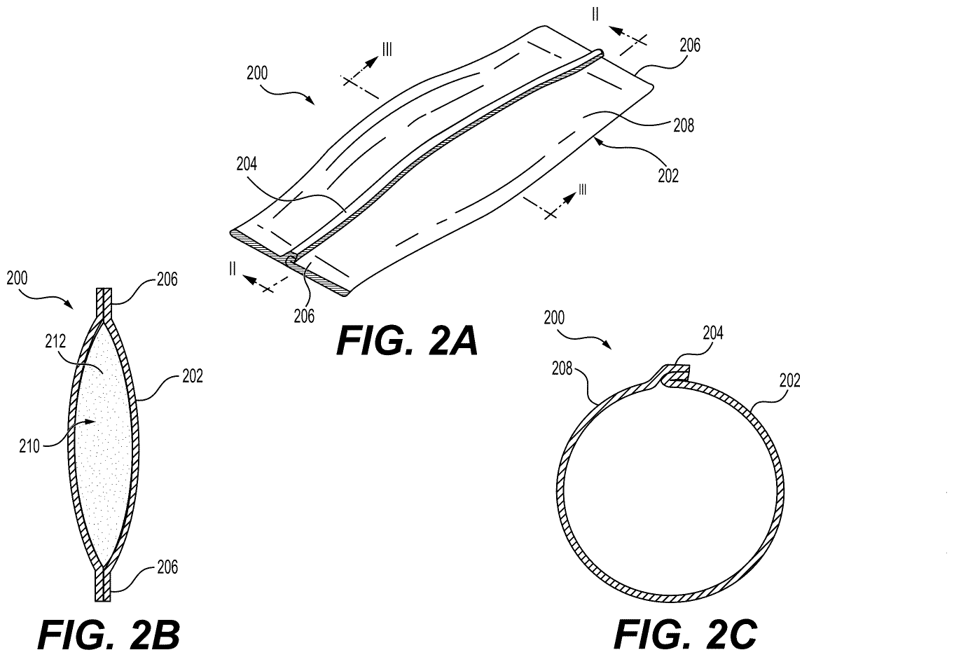

FIG. 2A is a perspective view of an oral pouch product according to at least one example embodiment.

FIG. 2B is a cross-sectional view of the oral pouch product along line II-II of FIG. 2A according to at least one example embodiment.

FIG. 2C is a cross-sectional view of the oral pouch product along line III-III of FIG. 2A according to at least one example embodiment.

In at least one example embodiment, as shown in FIGS. 2A-2C, an oral pouch product 200 is the same as that of FIGS. 1A-1B except that a pouch wrapper 202 includes one or more seals or seams, such as a longitudinal seal 204 and fin seals 206. The longitudinal seal 204 extends between the fin seals 206. In at least one example embodiment, as shown in FIG. 2C, the longitudinal seal 204 may include overlapping edge portions of the outer web 208 that are sealed together. In at least one example embodiment, the seals 204, 206 may have a dimension of less than about 1 mm. The sealing function can be accomplished by a food grade adhesive or by mutually sealing the overlapping edge portions, using thermal or sonic techniques.

In at least one example embodiment, as shown in FIG. 2B, opposing layers of an outer web 208, the longitudinal seal 204, and the fin seals 206 define an inner cavity 210 therebetween. A filler 212, which may be the same as that of FIGS. 1A-1B, may be held within the inner cavity 210. In at least one example embodiment, the filler 212 completely fills the interior cavity 210 of the oral pouch product 200. In other example embodiments, the filler 212 only partially fills the interior cavity 210 of the oral pouch product 200.

As illustrated in FIG. 2B, each fin seal 206 is formed by bringing together an inner surface of the outer web 208 of the pouch wrapper 202 and another section of the inner surface of the outer web 208 in a superposed relation to form one of the fin seal. The fin seal can then be sealed using any method such as detailed above to form the fin seal 206. Though not illustrated, in certain embodiments, integrated fin and longitudinal seals may be used, as would be recognized by the skilled artisan. By overlapping a fin seal, the oral pouch product 200 may be made more comfortable for insertion in the adult consumer's mouth because there are no loose, unsealed edges that may stick out and snag the consumer's mouth during enjoyment. In addition, integrated fin and longitudinal seals may be stronger to reduce and/or prevent breakage during manufacture, packaging, shipment, placement, and/or use of the oral pouch product 200.

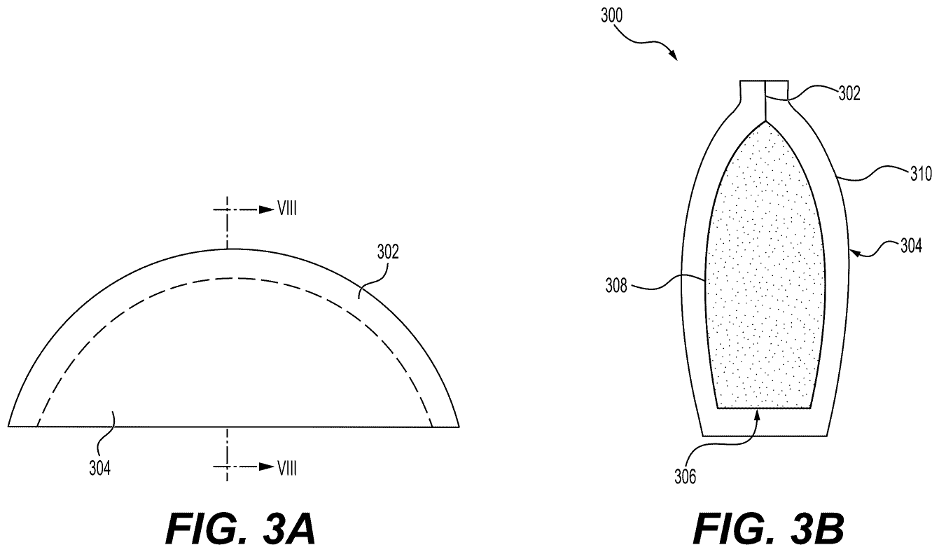

FIG. 3A is a side view of an oral pouch product according to at least one example embodiment.

FIG. 3B is a cross-sectional view along line VII-VII of the oral pouch product of FIG. 3A according at least one example embodiment.

In at least one example embodiment, as shown in FIGS. 3A-3B, an oral pouch product 300 is the same as that of FIGS. 1A-1B except that the oral pouch product 300 has a single seam or seal 302 along a pouch wrapper 304. In at least one example embodiment, as shown, the oral pouch product 300 has a half-moon shape. In some example embodiments, the pouch oral pouch product 300 has a D-shape, boomerang, crescent, or other shape.

In at least one example embodiment, the pouch wrapper 304 can be sealed along the seam or seal 302 to define an inner cavity 306 that is configured to contain or hold the filler 308, which is the same as described with respect to FIGS. 1A-1B. The single seam or seal 302 can be formed by bringing together an inner surface of an outer web 310 of the pouch wrapper 304 and another section of the inner surface of the outer web 310 in a superposed relation. The sealing function can be accomplished by a food grade adhesive or by mutual sealing the adjacent portions, using thermal or sonic techniques.

Three pouch compositions are prepared according to Table 1, below. Amounts in Table 1 are by weight percent.

TABLE 1
Pouch Compositions

| Component | Pouch 1 | Pouch 2 | Pouch 3 |
|---|---|---|---|
| Oil | 21.50% | 21.50% | 21.50% |
| Water | 2.25% | 12.50% | 25.00% |
| MCC | 58.75% | 48.50% | 36.00% |
| Other ingredients | 17.50% | 17.50% | 17.50% |
| Total | 100.00% | 100.00% | 100.00% |

The three pouch compositions are example embodiments and do not in any way limit the scope of the disclosure. For example, the water, oil, and insoluble material in the dry mixture (e.g. microcrystalline cellulose) may make up more than 80 weight percent of the pouch (e.g., more than 85 weight percent of the pouch, more than 90 weight percent of the pouch). The water, oil, and insoluble material in the dry mixture may make up less than 90 weight percent of the pouch (e.g., less than 85 weight percent of the pouch, less than 80 weigh percent of the pouch).

The filler of the pouch including the liquid mixture and the dry mixture may have a material bulk density of about 0.3 g/cm³ (e.g., between 0.25 g/cm³ and 0.5 g/cm³, between 0.3 g/cm³ and 0.45 g/cm³, between 0.3 g/cm³ and 0.4 g/cm³). For example, the bulk density of the filler may be greater than 0.25 g/cm³ (e.g., greater than 0.3 g/cm³, greater than 0.35 g/cm³, greater than 0.4 g/cm³). For example, the bulk density of the filler may be less than 0.45 g/cm³ (e.g., less than 0.4 g/cm³, less than 0.35 g/cm³, less than 0.3 g/cm³).

### Liquid Mixtures of Oil and Liquid Nicotine

In at least one example embodiment, the oral pouch product of FIGS. 1-3 includes a liquid mixture including oil, water, and nicotine as described below.

FIGS. 4A-4C depict chemical structures of nicotine in different forms.

Nicotine, or 3-(1-methyl-2-pyrrolidinyl) pyridine, is a tertiary amine. Under ambient conditions, nicotine is an oily, volatile, hygroscopic liquid. In this state, nicotine is a free base (non-protonated) and has the structure as shown in FIG. 4A. Free-base nicotine has a pKₐ value of about 8.

Nicotine may also be in a form of a complex or a salt. Nicotine complexes and salts may be provided in solid form, such as a powder. One example of a nicotine complex that is used in oral pouch products is nicotine polacrilex. In a salt, nicotine is mono-protonated, as shown in FIG. 4B, or di-protonated, as shown in FIG. 4C. Mono-protonated and di-protonated nicotine have lower pKₐ values than free-base nicotine. Nicotine salts may include nitrate, monotartrate, bitartrate, bitartrate dihydrate, salicylate, sulfate or bisulfate, phosphate or acid phosphate, acetate, lactate, succinate, maleate, fumarate, gluconate, saccharate, benzoate, methanesulfonate, hydrochloride, hydrobromide, hydroiodide, or any combination thereof.

Oral pouch products often include nicotine in the form of a complex or salt for reasons related to manufacturing, handling, and stability. However, nicotine is believed to more readily absorb in the buccal mucosa at higher pKₐ values. Accordingly, oral pouch products including nicotine in a protonated state may also include a pH adjuster so as to create a more basic environment in the oral pouch product.

In at least one example embodiment, an oral pouch product includes a liquid mixture of nicotine, water, and oil. The oil may include one or more triglycerides, one or more diglycerides, one or more monoglycerides, triacetin, triolein, trilinolein, vegetable oil, one or more partially-hydrogenated oils, or any combination thereof. The triglyceride may include one or more short-chain triglycerides (“SCT”), one or more medium chain triglycerides (“MCT”), one or more long chain triglycerides (“LCT”), or any combination thereof.

An oral pouch product including an oil phase may be configured to have increased buccal nicotine absorption. The nicotine may be liquid nicotine. At least a portion of the liquid nicotine may be dissolved in the oil and the water to form a solution of oil, water, and liquid nicotine.

A weight ratio of the oil to the nicotine in the liquid mixture may be greater than or equal to about 1:1 (e.g., greater than or equal to about 3:2, greater than or equal to about 2:1, greater than or equal to about 3:1, greater than or equal to about 4:1, greater than or equal to about 5:1, greater than or equal to about 6:1, greater than or equal to about 7:1, greater than or equal to about 8:1, greater than or equal to about 9:1, or greater than or equal to about 10:1). The weight ratio may be less than or equal to about 95:5 (e.g., less than or equal to about 9:1, less than or equal to about 8:1, less than or equal to about 7:1, less than or equal to about 6:1, less than or equal to about 5:1, less than or equal to about 4:1, less than or equal to about 3:1, or less than or equal to about 3:2). In at least one example embodiment, the weight ratio ranges from about 1:1 to about 9:1 (e.g., from about 3:2 to about 4:1, from about 3:1 to about 5:1, or about 4:1).

In at least one example embodiment, the oral pouch product may include additional oil beyond what is present in the liquid mixture. The additional oil may, in at least one example embodiment, be used as a plasticizer. A ratio of the oil to the liquid nicotine in the oral pouch product (i.e., both the liquid mixture and any additional oil) may be greater than or equal to about 1:1 (e.g., greater than or equal to about 3:2, greater than or equal to about 2:1, greater than or equal to about 3:1, greater than or equal to about 4:1, greater than or equal to about 5:1, greater than or equal to about 6:1, greater than or equal to about 7:1, greater than or equal to about 8:1, greater than or equal to about 9:1, greater than or equal to about 10:1, greater than or equal to about 15:1, greater than or equal to about 20:1, greater than or equal to about 25:1, greater than or equal to about 30:1, greater than or equal to about 40:1, greater than or equal to about 50:1, greater than or equal to about 60:1, greater than or equal to about 70:1, greater than or equal to about 75:1, or greater than or equal to about 80:1). The weight ratio of the triglyceride to the liquid nicotine may be less than or equal to about 100:1 (e.g., less than or equal to about 90:1, less than or equal to about 80:1, less than or equal to about 75:1, less than or equal to about 70:1, less than or equal to about 60:1, less than or equal to about 50:1, less than or equal to about 40:1, less than or equal to about 30:1, less than or equal to about 25:1, less than or equal to about 20:1, less than or equal to about 15:1, less than or equal to about 10:1, less than or equal to about 9:1, less than or equal to about 8:1, less than or equal to about 7:1, less than or equal to about 6:1, less than or equal to about 5:1, less than or equal to about 4:1, less than or equal to about 3:1, less than or equal to about 2:1, or less than or equal to about 3:2).

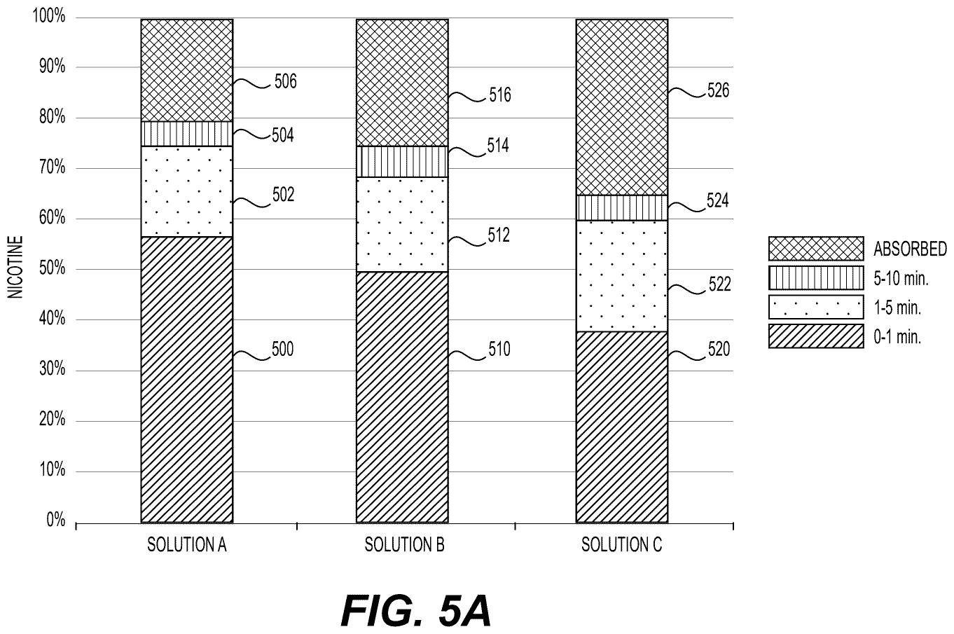

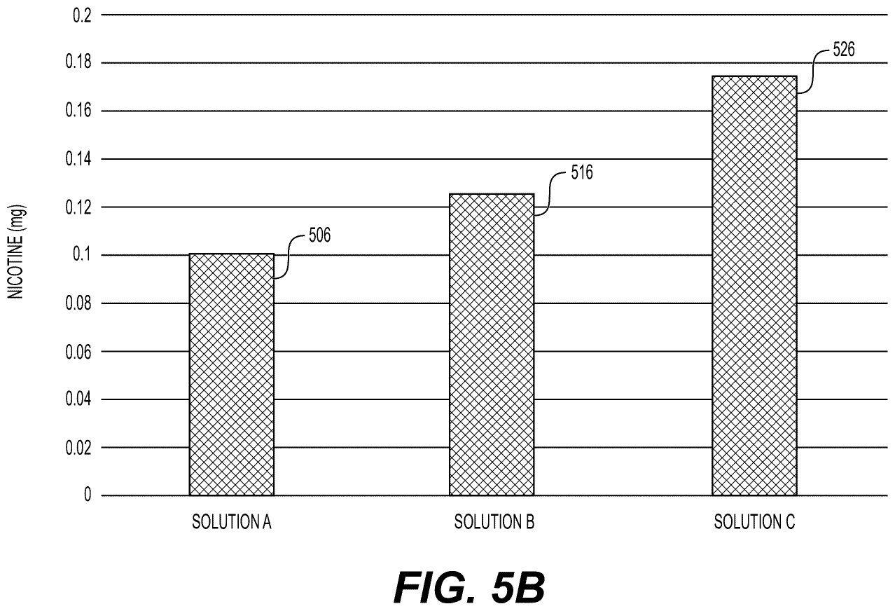

FIGS. 5A-5B are graphs depicting buccal nicotine disposition for different nicotine solutions.

In at least one example embodiment, providing the nicotine dissolved in an oil may facilitate increased buccal absorption. It is believed that providing the nicotine dissolved in the oil facilitates retention of at least a portion of the nicotine in its free-base state, regardless of the presence of a pH adjuster. At least one example embodiment does not include a pH adjuster.

As shown in FIGS. 5A-5B, three solutions are prepared according to Table 2, below. Amounts in Table 2 are by weight percent. Solution A includes 0.5 mg liquid nicotine in water having a pH of 7. Solution B includes 0.5 mg nicotine in water having a pH of 10. Solution C includes 0.5 mg nicotine in MCT oil.

TABLE 2
Solution Compositions by Weight Percent

| Component | Solution A | Solution B | Solution C |
|---|---|---|---|
| Nicotine | 0.050% | 0.050% | 0.050% |
| Propylene Glycol | 0.200% | 0.200% | — |
| MCT | — | — | 99.950% |
| Water | 99.732% | 99.746% | — |
| Citric Acid | 0.018% | — | — |
| Sodium Carbonate | — | 0.004% | — |

To determine the amount of nicotine absorbed, versus the amount not absorbed, each of Solutions A, B, and C is held in an oral cavity of an adult tobacco consumer. Five adult tobacco consumers participate. Expectorant samples are collected for each of the five adult tobacco consumers for each of the three solutions at three time intervals: 0-1 minute, 1-5 minutes, and 5-10 minutes. Each expectorant sample is analyzed to measure a weight of nicotine in the expectorant sample. The nicotine measured in the expectorant samples is necessarily not absorbed in the buccal mucosa. An amount of absorbed nicotine is calculated for each solution based on a difference between a known weight of nicotine in each solution and the measured amounts of nicotine in each of the expectorant samples for each solution, as described in greater detail below.

Solution A includes 0.5 mg of nicotine. A first solution A expectorant sample 500 is collected at 0-1 minute, and includes 57 weight percent of the 0.5 mg of nicotine. A second solution A expectorant sample 502 is collected at 1-5 minutes, and includes 18 weight percent of the 0.5 mg nicotine. A third solution A expectorant sample 504 is collected at 5-10 minutes, and includes 5 weight percent of the 0.5 mg of nicotine. Accordingly, a solution A absorbed nicotine 506 is calculated to be 20 weight percent (100%-57%-18%-5%=20%).

Solution B includes 0.5 mg of nicotine. A first solution B expectorant sample 510 is collected at 0-1 minute, and includes 50 weight percent of the 0.5 mg of nicotine. A second solution B expectorant sample 512 is collected at 1-5 minutes, and includes 19 weight percent of the 0.5 mg nicotine. A third solution B expectorant sample 514 is collected at 5-10 minutes, and includes 6 weight percent of the 0.5 mg of nicotine. Accordingly, a solution B absorbed nicotine 516 is calculated to be 25 weight percent (100%-50%-19%-6%=25%).

Solution C includes 0.5 mg of nicotine. A first solution C expectorant sample 520 is collected at 0-1 minute, and includes 38 weight percent of the 0.5 mg of nicotine. A second solution C expectorant sample 522 is collected at 1-5 minutes, and includes 22 weight percent of the 0.5 mg nicotine. A third solution C expectorant sample 524 is collected at 5-10 minutes, and includes 5 weight percent of the 0.5 mg of nicotine. Accordingly, a solution C absorbed nicotine 56 is calculated to be 35 weight percent (100%-38%-22%-5%=35%).

As shown by the differences in behavior of Solution A and Solution B, increasing a pH of an aqueous solution, as in Solution B, facilitates increased buccal nicotine absorption. For example, the increased pH of the aqueous environment may facilitate retention of nicotine in the free-base phase. As shown by the differences in behavior of Solutions A/B and Solution C, providing the nicotine in the MCT facilitates, as in Solution C, increased buccal nicotine absorption compared to providing nicotine in an aqueous phase. Moreover, as shown by the differences in behavior of Solution B and Solution C, providing the nicotine in the MCT facilitates, as in Solution C, increased buccal absorption compared to an aqueous phase including a pH adjuster. As shown in FIG. 2B, the amount of absorbed nicotine for Solution C is higher than the amount absorbed for each of Solutions A and B.

FIG. 6 is a graph depicting the time release of nicotine in for different oil/water solutions.

In at least one example embodiment, providing the water in a substantial amount, for example about 10 weight percent or more, in the liquid mixture may facilitate more rapid nicotine release.

As shown in FIG. 6, three filler mixtures were prepared according to Table 3, below. Amounts in Table 3 are by weight percent. The components of a first composition include 76.25 weight percent microcrystalline cellulose, 2.3 weight percent water, and 21.5 weight percent oil. The components of a second mixture include 66 weight percent microcrystalline cellulose, 12.5 weight percent water, and 21.5 weight percent oil. The components of a third mixture include 53.5 weight percent microcrystalline cellulose, 25 weight percent water, and 21.5 weight percent oil.

TABLE 3
Compositions by Weight Percent

| Component | Composition 1 | Composition 2 | Composition 3 |
|---|---|---|---|
| Oil | 21.50% | 21.50% | 21.50% |
| Water | 2.3% | 12.50% | 21.5% |
| MCC | 76.25% | 66% | 53.5% |

To determine the nicotine release profile was carried out using the U.S. Pharmacopeia flow-through cell dissolution apparatus 4 (USP-4). As can be seen from FIG. 6, Composition 2 and Composition 3 have a higher rate of nicotine release. It is believed that a greater weight fraction of water in the pouch facilitates a higher rate of nicotine release. For example, in at least one example embodiment a pouch may release at least 20 weight percent of the liquid nicotine based on a total weight of nicotine in the oral pouch product within 5 minutes upon placement in a consumer's mouth. In at least one example embodiment a pouch may release at least 30 weight percent nicotine within 10 minutes upon placement in a consumer's mouth. In at least one example embodiment a pouch may release at least 50 weight percent nicotine within 20 minutes upon placement in a consumer's mouth. In at least one example embodiment a pouch may release at least 60 weight percent nicotine within 30 minutes upon placement in a consumer's mouth. In at least one example embodiment a pouch may release at least 70 weight percent within 40 minutes upon placement in a consumer's mouth. In at least one example embodiment a pouch may release at least 80 weight percent nicotine within 60 minutes upon placement in a consumer's mouth. By including both water and oil in the liquid mixture, for example between 10 and 30 weight percent water and more than 20 weight percent oil, a higher rate of release and absorption by the consumer of nicotine may be achieved.

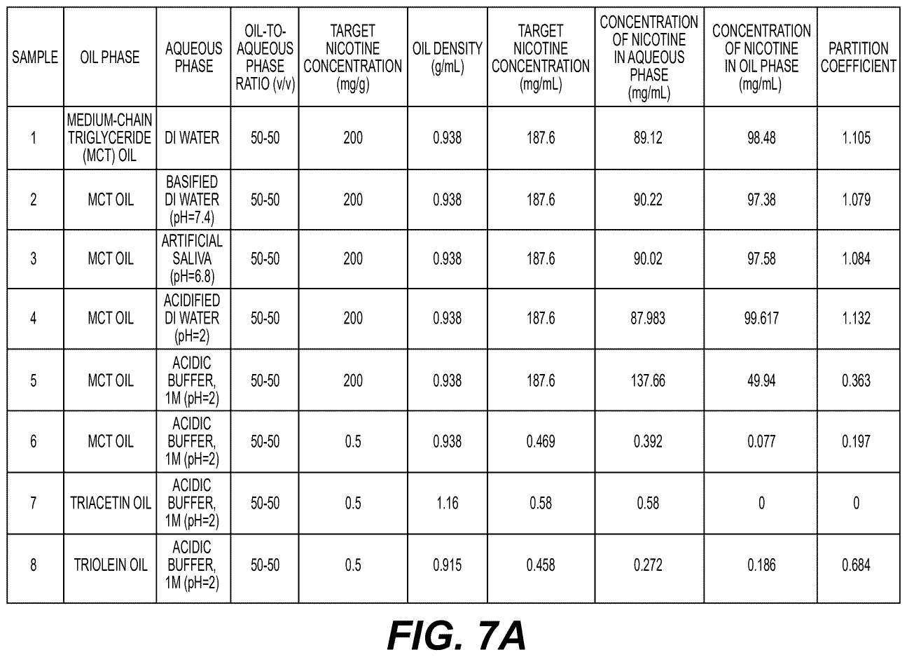

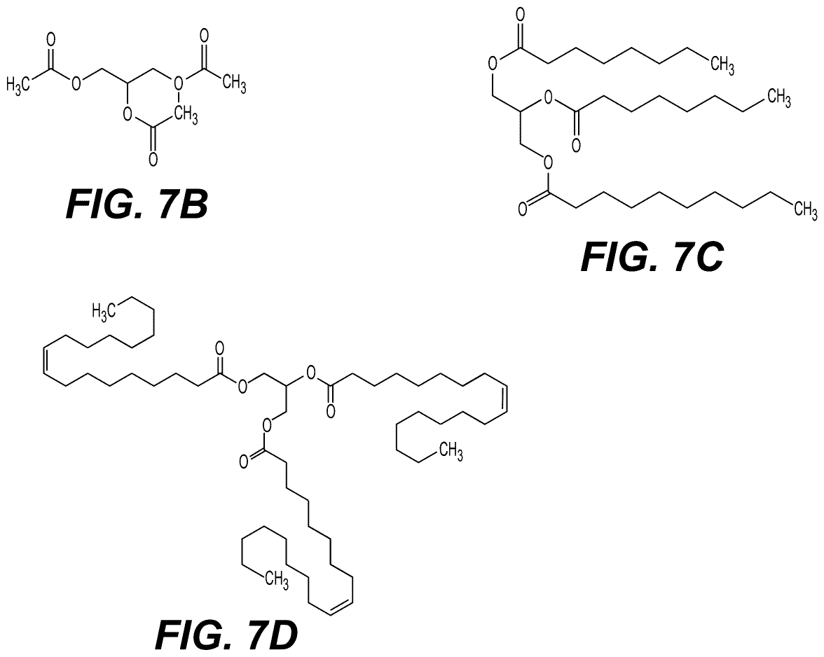

FIG. 7A is a table depicting partition coefficient data for nicotine in different oil and water phase combinations. FIG. 7B depicts a chemical structure of triacetin (C2). FIG. 7C depicts a chemical structure of MCT (C8-C10). FIG. 7D depicts a chemical structure of triolein (C18).

As shown in FIG. 7A, nicotine has solubility in both oil and water. Each of Samples 1-8 is prepared by and measured according to the following method. Liquid nicotine is added at the target nicotine concentration to a vessel containing an oil phase. The oil phase containing nicotine is mixed with an aqueous phase at a one-to-one volume ratio (v:v) using an orbital shaker at about 100 rotations per minute (rpm) for about 5 minutes to prepare a nicotine oil: aqueous phase mix. The nicotine oil:aqueous phase mix is allowed to sit at room temperature (about 25° C.) for about 24 hours. A concentration of nicotine in each of the oil phase and the nicotine phase is measured using a liquid chromatography method. A partition coefficient, which is a ratio of concentrations of a compound (i.e., nicotine) in two immiscible solvents (i.e., an oil phase and an aqueous phase) at equilibrium, is calculated for each sample. The partition coefficient is a comparison of the solubilities of the nicotine in the two liquid phases.

Samples 1-6 include MCT oil (CAS No. 73398-61-5) as an oil phase. Sample 7 includes triacetin oil as an oil phase. Sample 8 includes triolein oil as an oil phase. Sample 1 includes deionized (DI) water as an aqueous phase. Sample 2 includes basified DI water having a pH of 7.4 as an aqueous phase. Sample 3 includes artificial saliva having a pH of 6.8 as an aqueous phase. Artificial saliva simulates mucus conditions. Sample 4 includes acidified DI water having a pH of 2 as an aqueous phase. Samples 5-8 include 1M acetic acid pH adjuster having a pH of 2 as an aqueous phase, which simulates stomach conditions.

In each of Samples 5-8, a partition coefficient is less than 1, indicating a higher solubility of nicotine in the acetic acid pH adjuster than the respective oil phase. This is believed to be caused by protonation of the nicotine and higher electrostatic interactions. In Samples 1-4, a partition coefficient is greater than 1, indicating a higher solubility in the MCT oil than the respective aqueous phase. However, in every sample, at least a portion of the nicotine is dissolved in the aqueous phase. Accordingly, when water is present in a liquid mixture, at least a portion of the nicotine will be dissolved in the water phase.

In at least one example embodiment, the oral pouch product includes water. The oral pouch product may include water in an amount greater than or equal to about 1 weight percent (e.g., more than or equal to about 8 weight percent, more than or equal to about 10 weight percent, more than or equal to about 15 weight percent, more than or equal to about 20 weight percent, more than or equal to about 25 weight percent, more than or equal to about 30 weight percent.

In at least one example embodiment, the liquid mixture includes water in amount less than or equal to about 35 weight percent (e.g., less than or equal to about 30 weight percent, less than or equal to about 25 weight percent, less than or equal to about 20 weight percent, less than or equal to about 20 weight percent, or less than or equal to about 15 weight percent). In at least one example embodiment, the liquid mixture includes water in an amount greater than or equal to about 1 weight percent (e.g., greater than or equal to about 5 weight percent, greater than or equal to about 10 weight percent, greater than or equal to about 15 weight percent, greater than equal to about 20 weight percent, greater than or equal to about 25 weight percent, or greater than equal to about 30 weight percent).

As noted above, in at least one example embodiment, the nicotine may be liquid nicotine. Greater than or equal to about 50 weight percent of the nicotine may be free base nicotine (e.g., greater than or equal to about 55 weight percent, greater than or equal to about 60 weight percent, greater than or equal to about 65 weight percent, greater than or equal to about 70 weight percent, greater than or equal to about 75 weight percent, greater than or equal to about 80 weight percent, greater than or equal to about 85 weight percent, greater than or equal to about 90 weight percent, greater than or equal to about 95 weight percent, greater than or equal to about 98 weight percent, greater than or equal to about 99 weight percent). In at least one example embodiment, greater than 80 weight percent of the nicotine is free-base nicotine.

In at least one example embodiment, the nicotine is tobacco-derived nicotine, synthetic nicotine, or both tobacco-derived nicotine and synthetic nicotine. In at least one example embodiment, the oral pouch product includes the nicotine in an amount greater than or equal to about 0.1 mg (e.g., greater than or equal to about 1 mg, greater than or equal to about 2 mg, greater than or equal to about 4 mg, greater than or equal to about 6 mg, greater than or equal to about 8 mg, greater than or equal to about 10 mg, greater than or equal to about 12 mg). The oral pouch product may include the nicotine in an amount less than or equal to about 14 mg (e.g., less than or equal to about 12 mg, less than or equal to about 10 mg, less than or equal to about 8 mg, less than or equal to about 6 mg, less than or equal to about 4 mg, less than or equal to about 2 mg, or less than or equal to about 1 mg). In at least one example embodiment, the oral pouch product includes the liquid nicotine in an amount ranging from 0.1 mg to about 14 mg (e.g., ranging from about 2 mg to about 10 mg, ranging from about 4 mg to about 8 mg, or ranging from about 5 mg to about 6 mg).

As discussed above, the oil may include one or more monoglycerides, one or more diglycerides, one or more triglycerides, triacetin, triolein, trilinolein, vegetable oil, one or more partially-hydrogenated oils, or any combination thereof. The triglyceride may include one or more LCTs, one or more MCTs, one or more SCTs, or any combination thereof. In at least one example embodiment, the oil includes one or more monoglyceride, one or more diglycerides, one or more triglycerides, or any combination thereof. In at least one example embodiment, the oil includes one or more triglycerides. In at least one example embodiment, the triglyceride includes MCT. In at least one example embodiment, the liquid mixture further includes triacetin, triolein, trilinolein, vegetable oil, a partially-hydrogenated oil, or any combination thereof.

In at least one example embodiment, the oral pouch product includes the oil (e.g., triglyceride) in an amount greater than or equal to about 1 weight percent (e.g., greater than or equal to about 2 weight percent, greater than or equal to about 3 weight percent, greater than or equal to about 4 weight percent, greater than or equal to about 5 weight percent, greater than or equal to about 10 weight percent, greater than or equal to about 15 weight percent, greater than or equal to about 20 weight percent, greater than or equal to about 25 weight percent, greater than or equal to about 30 weight percent, greater than or equal to about 40 weight percent, greater than or equal to about 50 weight percent, greater than or equal to about 60 weight percent, greater than or equal to about 70 weight percent, greater than or equal to about 80 weight percent, or greater than or equal to about 90 weight percent). In at least one example embodiment, the oral pouch product may include the oil (e.g., triglyceride) in an amount less than or equal to about 95 weight percent (e.g., less than or equal to about 90 weight percent, less than or equal to about 80 weight percent, less than or equal to about 70 weight percent, less than or equal to about 60 weight percent, less than or equal to about 50 weight percent, less than or equal to about 40 weight percent, less than or equal to about 30 weight percent, less than or equal to about 20 weight percent, less than or equal to about 15 weight percent, less than or equal to about 10 weight percent, less than or equal to about 7 weight percent, or less than or equal to about 5 weight percent). In at least one example embodiment, the oral pouch product includes the oil (e.g., triglyceride) in an amount ranging from 10 weight percent to 50 weight percent. In at least one example embodiment, the oral pouch product is a solid format including the oil (e.g., triglyceride) at less than or equal to about 50 weight percent.

### Methods of Manufacturing an Oral Pouch Product

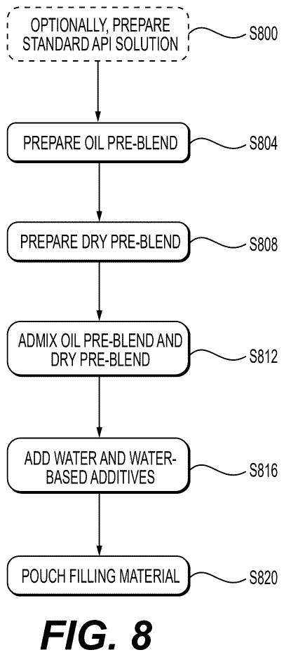

FIG. 8 is a flowchart depicting a method of manufacturing a pouch product according to at least one example embodiment.

As shown in FIG. 8, a method of preparing a filler may optionally include preparing a standard API solution at S800. The standard API solution may include an API, such as liquid nicotine, dissolved in oil, such as MCT, at a desired (or alternatively, predetermined) weight ratio. In at least one example embodiment, the desired weight ratio is greater than or equal to about 20:80 (1:4) (e.g., greater than or equal to about 25:75, greater than or equal to about 30:70, greater than or equal to about 35:65, greater than or equal to about 40:60 (2:3), greater than or equal to about 45:55, greater than or equal to about 50:50, greater than or equal to about 55:45, greater than or equal to about 60:40, greater than or equal to about 65:35, greater than or equal to about 70:30, or greater than or equal to about 75:25). The desired weight ratio may be less than or equal to about 80:20 (e.g., less than or equal to about 75:25, less than or equal to about 70:30, less than or equal to about 65:35, less than or equal to about 60:40, less than or equal to about 55:45, less than or equal to about 50:50, less than or equal to about 45:50, less than or equal to about 40:60, less than or equal to about 35:65, less than or equal to about 30:70, or less than or equal to about 25:75).

At S804, the method may include preparing an oil pre-blend. In at least one example embodiment, the oil prep-blend includes API, oil, and antioxidant. In at least one example embodiment, the oil pre-blend may be prepared by providing the standard API solution in an amount suitable to achieve a desired API content in the pouch product (i.e., more standard API solution to prepare a higher API dose product). Antioxidant, such as liquid antioxidant, may also be added to the oil and API. An amount of antioxidant may be selected based on the amount of API.

At S808, the method may include preparing a dry pre-blend. In at least one example embodiment, preparing the dry pre-blend includes admixing the insoluble material, the water-soluble filler, the pH adjuster, dry flavorant, dry antioxidant, plant material (e.g., cannabis and/or tobacco plant material), the emulsifier and/or surfactant, and/or other dry additives.

At S812, the method may include admixing the oil pre-blend and the dry pre-blend. In at least one example embodiments, one or more of water activity, pH, oven volatiles, bulk density, dissolution, flowability, and API content are monitored at one or more points in the process of preparing the oral pouch product. Admixing the oil and dry pre-blends may include providing the oil pre-blend in an atomized spray. The ingredients may be admixed, such as in a ribbon blender. A weight ratio of dry to liquid ingredients may be as described above in the discussion accompanying FIGS. 1A-1B.

At S816, the method may include adding water and water-based additives. Water-based additives, such as flavorant, may be added by spraying and or pouring, optionally while admixing, such as in the ribbon blender. In at least one example embodiment, the dry pre-blend is continuously stirred while the liquid pre-blend is added at S812 and the water-based additives are added at S816.

At S820, the method may include pouching the filler. Pouching the filler may generally include forming a wrapper into an open pouch using a vertical or horizontal fill machine and filling the open pouch with the filler. The pouch may then be sealed to contain the filler and form the oral pouch product. In at least one example embodiment, a series of oral pouch products are formed with a space between seals of adjacent pouch products and then cut apart to form individual pouch products. For instance, the oral pouch product may be cut with a die at a location between adjacent seals to form a soft edge on each pouch product. Alternatively, a first strip of pouch wrapper material can be advanced along a feed path, filler can be placed on the strip, a second strip can be placed over the first strip, a sealing die can be used to press the strips together and form a seam such as a heat seal or adhesive seal around the filling, and a cuffing die can be used to cut the first and second strips outwardly of the seam to form the soft edge.

However, the method is not limited to the order depicted in FIG. 8. For example, the water and water-based additives may be added to the oil pre-blend prior to being admixed with the dry pre-blend.

While some example embodiments have been disclosed herein, it should be understood that other variations may be possible. Such variations are not to be regarded as a departure from the spirit and scope of the present disclosure, and all such modifications as would be obvious to one skilled in the art are intended to be included within the scope of the following claims.

Illustrative embodiment 1. An oral pouch product comprising: a wrapper defining a cavity; and a filler in the cavity, the filler including, a dry mixture, and a liquid mixture including, an oil, and water in an amount of 10 weight percent or more based on a weight of the filler.

Illustrative embodiment 2. The oral pouch product of Illustrative embodiment 1, wherein a water content is between 10 and 30 weight percent based on the weight of the filler.

Illustrative embodiment 3. The oral pouch product of Illustrative embodiment 2, wherein the water content is between 20 and 30 weight percent based on the weight of the filler.

Illustrative embodiment 4. The oral pouch product of any of illustrative embodiments 1 through 3, further comprising: at least one additive.

Illustrative embodiment 5. The oral pouch product of Illustrative embodiment 4, wherein the at least one additive includes liquid nicotine.

Illustrative embodiment 6. The oral pouch product of illustrative embodiment 5, wherein the oral pouch product is configured to release at least 30 weight percent of the liquid nicotine based on a total weight of nicotine in the oral pouch product within 10 minutes upon placement in a mouth.

Illustrative embodiment 7. The oral pouch product of any of Illustrative embodiments 4 through 6, wherein the at least one additive includes a sweetener, a pH adjuster, a flavorant, a humectant, or any combination thereof.

Illustrative embodiment 8. The oral pouch product of any of Illustrative embodiments 1 through 7, wherein a density of the filler is about 0.3 g/cm³.

Illustrative embodiment 9. The oral pouch product of any of Illustrative embodiments 1 through 8, wherein the oil includes a medium chain triglyceride.

Illustrative embodiment 10. The oral pouch product of any of Illustrative embodiments 1 through 9, wherein the dry mixture includes a cellulosic material.

Illustrative embodiment 11. The oral pouch product of Illustrative embodiment 10, wherein the dry mixture includes microcrystalline cellulose.

Illustrative embodiment 12. The oral pouch product of any of Illustrative embodiments 1 through 11, wherein the filler includes between 30 weight percent to 60 weight percent dry mixture.

Illustrative embodiment 13. The oral pouch product of any of Illustrative embodiments 1 through 12, wherein the filler includes more than 20 weight percent oil.

Illustrative embodiment 14. The oral pouch product of Illustrative embodiment 13, wherein the filler includes between 10 weight percent and 30 weight percent water based on the weight of the filler.

Illustrative embodiment 15. The oral pouch product of any of Illustrative embodiments 1 through 14, wherein the filler includes, a medium chain triglyceride; water; microcrystalline cellulose; and liquid nicotine.

Illustrative embodiment 16. The oral pouch product of any of Illustrative embodiments 1 through 15, wherein the filler includes, more than 20 weight percent oil; between 10 weight percent and 30 weight percent water; and between 30 weight percent and 60 weight percent dry mixture.

Illustrative embodiment 17. The oral pouch product of any of Illustrative embodiments 1 through 16, wherein the dry mixture is insoluble in the liquid mixture.

Illustrative embodiment 18. An oral pouch product comprising: a wrapper defining a cavity; and a filler in the cavity, the filler including, a dry mixture including between 30 weight percent and 60 weight percent microcrystalline cellulose based on a weight of the filler, and a liquid mixture including more than 20 weight percent medium chain triglyceride based on the weight of the filler, water in an amount between 10 weight percent and 30 weight percent based on the weight of the filler, and liquid nicotine.

Illustrative embodiment 19. The oral pouch product of Illustrative embodiment 18, wherein the oral pouch product is configured to release at least 30 weight percent of the liquid nicotine based on a total weight of nicotine in the oral pouch product within 10 minutes upon placement in a mouth.

Illustrative embodiment 20. The oral pouch product of any of Illustrative embodiments 18 or 19, further comprising: at least one additive including a sweetener, a pH adjuster, a flavorant, a humectant, or any combination thereof.

Illustrative embodiment 21. A method of preparing a filler composition for oral pouch products, the method comprising: mixing an active ingredient solution with oil to form an oil pre-blend; preparing a dry pre-blend including an insoluble material and a dry flavorant; combining the oil pre-blend with the dry pre-blend to form a first mixture; and adding a water solution including a water-based flavorant into the first mixture to form a filler, water in the water solution is included in an amount of 10 weight percent or more based on a weight of the filler.

Illustrative embodiment 22. The method of Illustrative embodiment 21, wherein the oil pre-blend additionally includes an antioxidant.

Illustrative embodiment 23. The method of any of Illustrative embodiments 21 or 22, wherein the dry pre-blend additionally includes a water-soluble filler.

Illustrative embodiment 24. The method of any of Illustrative embodiments 21 through 23, wherein the dry pre-blend includes at least one of a pH adjuster, a dry antioxidant, plant material, an emulsifier, a surfactant, or any combination thereof.

Illustrative embodiment 25. The method of any of Illustrative embodiments 21 through 25, wherein the oil pre-blend is combined with the dry pre-blend in the form of an atomized spray.

Illustrative embodiment 26. The method of any of Illustrative embodiments 21 through 25, further comprising: filling a pouch with the filler; and sealing the pouch to contain the filler and form an oral pouch product.

Illustrative embodiment 27. The method of any of Illustrative embodiments 21 through 26, further comprising: forming the active ingredient solution by dissolving an active ingredient in oil in an amount greater than or equal to a weight ratio of 20:80 active ingredient to oil and less than our equal to a weight ratio of 80:20 active ingredient to oil.

Illustrative embodiment 28. The method of any of Illustrative embodiments 21 through 27, wherein the oil is a medium chain triglyceride.

Illustrative embodiment 29. The method of any of Illustrative embodiments 21 through 28, wherein the oil is included in an amount greater than or equal to 20 weight percent of the filler.

Illustrative embodiment 30. The method of any of Illustrative embodiments 21 through 29, wherein the dry pre-blend is included in an amount greater than or equal to 30 weight percent of the filler and less than or equal to 60 weight percent of the filler.

Although described with reference to specific examples and drawings, modifications, additions and substitutions of example embodiments may be variously made according to the description by those of ordinary skill in the art. For example, the described techniques may be performed in an order different with that of the methods described, and/or elements such as the described system, architecture, devices, circuit, and the like, may be connected or combined to be different from the above-described methods, or results may be appropriately achieved by other elements or equivalents.

We claim:

1. An oral pouch product comprising:
a wrapper defining a cavity; and
a filler in the cavity, the filler including,
a dry mixture, and
a liquid mixture including,
an oil, and
water in an amount of 10 weight percent or more based on a weight of the filler.

2. The oral pouch product of claim 1, wherein a water content is between 10 and 30 weight percent based on the weight of the filler.

3. The oral pouch product of claim 2, wherein the water content is between 20 and 30 weight percent based on the weight of the filler.

4. The oral pouch product of claim 1, further comprising:
at least one additive.

5. The oral pouch product of claim 4, wherein the at least one additive includes liquid nicotine.

6. The oral pouch product of claim 5, wherein the oral pouch product is configured to release at least 30 weight percent of the liquid nicotine based on a total weight of nicotine in the oral pouch product within 10 minutes upon placement in a mouth.

7. The oral pouch product of claim 4, wherein the at least one additive includes a sweetener, a pH adjuster, a flavorant, a humectant, or any combination thereof.

8. The oral pouch product of claim 1, wherein a density of the filler is about 0.3 g/cm³.

9. The oral pouch product of claim 1, wherein the oil includes a medium chain triglyceride.

10. The oral pouch product of claim 1, wherein the dry mixture includes a cellulosic material.

11. The oral pouch product of claim 10, wherein the dry mixture includes microcrystalline cellulose.

12. The oral pouch product of claim 1, wherein the filler includes between 30 weight percent to 60 weight percent dry mixture.

13. The oral pouch product of claim 1, wherein the filler includes more than 20 weight percent oil.

14. The oral pouch product of claim 13, wherein the filler includes between 10 weight percent and 30 weight percent water based on the weight of the filler.

15. The oral pouch product of claim 1, wherein the filler includes,
a medium chain triglyceride;
water;
microcrystalline cellulose; and
liquid nicotine.

16. The oral pouch product of claim 1, wherein the filler includes,
more than 20 weight percent oil;
between 10 weight percent and 30 weight percent water; and
between 30 weight percent and 60 weight percent dry mixture.

17. The oral pouch product of claim 1, wherein the dry mixture is insoluble in the liquid mixture.

18. An oral pouch product comprising:
a wrapper defining a cavity; and
a filler in the cavity, the filler including,
a dry mixture including between 30 weight percent and 60 weight percent microcrystalline cellulose based on a weight of the filler, and
a liquid mixture including,
more than 20 weight percent medium chain triglyceride based on the weight of the filler,
water in an amount between 10 weight percent and 30 weight percent based on the weight of the filler, and
liquid nicotine.

19. The oral pouch product of claim 18, wherein the oral pouch product is configured to release at least 30 weight percent of the liquid nicotine based on a total weight of nicotine in the oral pouch product within 10 minutes upon placement in a mouth.

20. A method of preparing a filler composition for oral pouch products, the method comprising:
mixing an active ingredient solution with oil to form an oil pre-blend;
preparing a dry pre-blend including an insoluble material and a dry flavorant;
combining the oil pre-blend with the dry pre-blend to form a first mixture; and
adding a water solution including a water-based flavorant into the first mixture to form a filler, water in the water solution is included in an amount of 10 weight percent or more based on a weight of the filler.
